SEQUENCE LISTING (11 sequences)

SEQ ID NO: 1 - DNA, 1396 bp, Homo sapiens
agccaccatcgaggaaggggcatgtgctggacgcggacacatgatccgagggaccctgctgggtggaactaagaaagtccagcagactgtgcatgctcctgtccccactcacaggcccacgcagcgaggggggcccctcttctgtgtgcgtctggaaggtcgctgcccagggaggaaatgcctttaaccggcgtggggccgggcagctgcaggaggcagatcatccgggctctgtgcctcttgctacttctcctccacgccggctctgccaagaatatctggaaacgggcattgcctgcgaggctggccgagaaatcccgtgccgaggaggcgggcgcgcccggcggcccccggcagccccgagcagaccgctgcccgccgcctccgcggacgctgccccccggcgcctgccaggccgcgcgctgtcaggcggactccgagtgcccgcggcaccggcgctgctgctacaacggatgcgcctacgcctgcctagaagctgtgccgcccccgccagtcttagactggctggtgcagccgaaacctcgatggcttggtggcaatggctggctcctggatggccctgaggaggtgttacaagcagaggcgtgcagcaccacggaggatggggccgaacccctgctctgtccctcgggctatgagtgccacatcctgagcccaggtgacgtggccgaaggtatccccaaccgtgggcagtgcgtcaagcagcgccggcaagcagatgggcgaatcctacgacacaaactttacaaagaatatccagaaggtgactcaaagaatgtggcagaacctggaaggggacaacagaagcactttcagtaaagcaacggcaagcagctaggttgcaagaacattcctctactttctgctaagccttggaaacagttgggaaaagtagtttgaccctcacagttcacattcagctcagcagagcaagaccccagagatgcttagagacaggacacctggccctcaaacccagtttggcccagcctggttgggtgactttgtgggagccacttaacagctctgggtccctgttttaccatcctgggagcaaggccctgcagctccacgagacctttaccccgggaagaagccgccgcccatgaaagcatttctgaagcccctttctaagacaaggctcagcatcttgatatttttgacagattcctcccaagtctggctctgggaggtatgtacccatctcaaatgttcccaagataaattcatccttcaggaaatggaaatgaacttgcttactaatgtgtgattcctagttgtagccaccggatgtgctgaggcctaaatgttagcaggtgggaggaggccacagaacaataaaaacaaccaaataagaaaaaaaaaaaaaaaaaaa

SEQ ID NO: 2 - protein, 220 aa, Homo sapiens
MPLTGVGPGSCRRQIIRALCLLLLLLHAGSAKNIWKRALPARLAEKSRAEEAGAPGGPRQPRADRCPPPPRTLPPGACQAARCQADSECPRHRRCCYNGCAYACLEAVPPPPVLDWLVQPKPRWLGGNGWLLDGPEEVLQAEACSTTEDGAEPLLCPSGYECHILSPGDVAEGIPNRGQCVKQRRQADGRILRHKLYKEYPEGDSKNVAEPGRGQQKHFQ

SEQ ID NO: 3 - protein, 220 aa, Homo sapiens [sequence omitted]

SEQ ID NO: 4 - protein, 16 aa, Artificial Sequence
  Synthetic - Peptide
EAGAPGGPRQPRADRC

SEQ ID NO: 5 - protein, 16 aa, Artificial Sequence
  Synthetic - Peptide
CKNVAEPGRGQQRHFQ

SEQ ID NO: 6 - DNA, 21 bp, Artificial Sequence
  Synthetic - siRNA
ggugacucaaagaauguggtt

SEQ ID NO: 7 - DNA, 21 bp, Artificial Sequence
  Synthetic - siRNA
ccacauucuuugagucacctt

SEQ ID NO: 8 - DNA, 21 bp, Artificial Sequence
  Synthetic - siRNA
ggcucagcaucuugauauutt

SEQ ID NO: 9 - DNA, 21 bp, Artificial Sequence
  Synthetic - siRNA
aauaucaagaugcugagcctt

SEQ ID NO: 10 - DNA, 21 bp, Artificial Sequence
  Synthetic - siRNA
ugcugacuccaaagcucugdt

SEQ ID NO: 11 - DNA, 21 bp, Artificial Sequence
  Synthetic - siRNA
cagagcuuuggagucagcadt

(19) United States
(12) Patent Application Publication
(10) Pub. No.: US 2011/0212076 A1
(43) Pub. Date: Sep. 1, 2011

(54) Viral Therapeutic

(75) Inventors: Annapurna Vyakarnam, London (GB); David R. Rowley, Houston, TX (US)

(73) Assignees: KING'S COLLEGE LONDON, London (GB); BAYLOR COLLEGE OF MEDICINE, Houston, TX (US)

(21) Appl. No.: 12/682,978

(22) PCT Filed: Oct. 15, 2008
(86) PCT No.: PCT/GB08/03479

§ 371 Date: Apr. 14, 2010

Related U.S. Application Data
(60) Provisional application No. 60/998,953, filed on Oct. 15, 2007.
(60) Provisional application No. 60/998,951, filed on Oct. 15, 2007.

(51) Int. Cl. A61K 39/395; C12N 5/071; A61K 31/7088; G01N 33/53; A61P 31/12; A61P 31/16; A61P 37/04

(52) U.S. Cl. 424/130.1; 435/325; 514/44 A; 435/7.24

(57) ABSTRACT

The invention provides a method of inhibiting viral infection of a mammalian cell, said method comprising reducing or inhibiting ps20 polypeptide expressed by said cell. Suitably ps20 is inhibited by contacting said cell with an antibody capable of binding to ps20 polypeptide. Suitably said antibody is ps20 neutralising antibody. The invention also provides antibody capable of binding ps20 polypeptide, siRNA targeted to a transcript encoding ps20 polypeptide, or antisense ps20 polynucleotide for use as a medicament for viral infection. The invention also provides a method of identifying an agent for inhibiting a viral infection, comprising determining level of ps20 expression in first and second samples, the first contacted with test agent; and comparing the level of ps20 expression in said first and second samples; wherein lower level of ps20 expression in said first sample relative to said second sample identifies test agent as an agent for inhibiting a viral infection.

### FIELD OF THE INVENTION

The invention relates to compositions and methods for the treatment, prevention and amelioration of viral diseases.

### BACKGROUND OF THE INVENTION

Viruses are ever-present pathogens capable of producing primary, latent, and recurrent infections which contribute to a variety of diseases. There is a critical need for antiviral drugs for the efficient management of viral infections. Viruses may rely on host cell, factors for infection, replication and/or pathogenesis and they represent potential therapeutic targets. Of particular interest are host cell factors that mediate virus entry or facilitate replication and assembly.

Studying viral entry and viral spread in mammalian systems is a challenging technical field. Despite a number advances in understanding this subject, there is still many enigmatic elements to the biology and many areas of active investigation. Whilst some of the factors in viral entry and viral spread are reasonably well understood in the art, others remain obscure. This has been a problem in the art.

### SUMMARY OF THE INVENTION

The present inventors have discovered a new biological function for the cell surface and secreted protein ps20. In particular, the inventors have pinpointed this protein as important for entry of free virus into mammalian cells. In addition, the inventors have also shown a key role for this protein in cell-to-cell viral spread. In developing these findings, the inventors have created numerous reagents in connection with the ps20 protein, and have identified numerous epitopes and regions of ps20 which can be targeted in order to neutralise the protein. Furthermore, the inventors go on to experimentally demonstrate that reagents targeted to ps20 have genuine therapeutic value, and are able to suppress viral entry, and are further able to suppress cell-to-cell viral spread. As well as applying these findings to diverse mammalian viruses, the inventors also provide substantial experimental demonstration of the value of these therapeutic approaches in connection with human immunodeficiency virus (HIV). The present invention is based on these remarkable findings.

Thus in one aspect the invention provides a method of inhibiting viral infection of a mammalian cell, said method comprising reducing or inhibiting ps20 polypeptide expressed by said cell.

Reduction may be by removal or more suitably by suppression of expression/production of new ps20 polypeptide. Examples are application of siRNA and/or antisense to suppress ps20 expression.

Inhibition may be by any suitable means and is preferably by use of immunological reagent(s) which target ps20 for example by binding to ps20. Preferred are antibodies to ps20 such as neutralizing antibodies to ps20. Inhibition is suitably neutralization of ps20 polypeptide.

Thus suitably ps20 is inhibited by contacting said cell with an antibody capable of binding to ps20 polypeptide. More suitably said antibody is a ps20 neutralising antibody.

Suitably said antibody comprises an antibody to one or more neutralising epitope(s) on ps20. We disclose numerous neutralising epitopes based on the fact that certain peptides disclosed herein can enhance HIV infection and conversely that antibodies to at least three of these epitopes (e.g. the IG7 antibody and the polyclonal antibodies) can block HIV infection. If a skilled worker wishes to determine whether or not a particular ps20 antibody is a neutralising antibody, they may simply test it according to the examples set out below e.g. assess the capacity of the test antibody to neutralise ps20, thereby blocking HIV infection.

Exemplary neutralising antibodies are those which bind ps20 peptide(s) as set out in the examples. Further exemplary neutralising antibodies are as disclosed herein such as IG7 monoclonal antibody.

The invention may also relate to an antibody such as a ps20 neutralising antibody raised against one or more of the ps20 peptide(s) as set out in the examples. In particular, suitably said antibody is raised against and/or reacts with the ‘555’ peptide i.e. a peptide comprising amino acids 21-35 of the ps20 sequence.

Suitably said antibody is a single chain antibody

Throughout the aspects and embodiments of this invention, suitably the virus or viral infection is human immunodeficiency virus or human immunodeficiency virus infection.

In another aspect, the invention relates to an antibody capable of binding ps20 polypeptide for use as a medicament, suitably for viral infection.

In another aspect, the invention relates to siRNA targeted to a transcript encoding a ps20 polypeptide for use as a medicament, suitably for viral infection.

In another aspect, the invention relates to antisense ps20 polynucleotide for use as a medicament, suitably for viral infection.

In another aspect, the invention relates to use of antibody capable of binding ps20 polypeptide for the manufacture of a medicament for viral infection.

In another aspect, the invention relates to use of siRNA targeted to a transcript encoding a ps20 polypeptide for the manufacture of a medicament for viral infection.

In another aspect, the invention relates to use of antisense ps20 polynucleotide for the manufacture of a medicament for viral infection.

In another aspect, the invention relates to antibody capable of binding ps20 polypeptide for use in the treatment of viral infection.

In another aspect, the invention relates to siRNA targeted to a transcript encoding a ps20 polypeptide for use in the treatment of viral infection.

In another aspect, the invention relates to antisense ps20 polynucleotide for use in the treatment of viral infection.

In another aspect, the invention relates to use of antibody capable of binding ps20 polypeptide for the manufacture of a medicament for human immunodeficiency virus infection.

In another aspect, the invention relates to use of siRNA targeted to a transcript encoding a ps20 polypeptide for the manufacture of a medicament for human immunodeficiency virus infection.

In another aspect, the invention relates to use of antisense ps20 polynucleotide for the manufacture of a medicament for human immunodeficiency virus infection.

In another aspect, the invention relates to antibody capable of binding ps20 polypeptide for use in the treatment of human immunodeficiency virus infection.

In another aspect, the invention relates to siRNA targeted to a transcript encoding a ps20 polypeptide for use in the treatment of human immunodeficiency virus infection.

In another aspect, the invention relates to antisense ps20 polynucleotide for use in the treatment of human immunodeficiency virus infection.

In another aspect, the invention relates to a method of treating or preventing viral infection in a subject, said method comprising reducing or inhibiting ps20 polypeptide in said subject. Suitably said method comprises administering one or more of

(i) an antibody capable of binding to ps20 polypeptide; or

(ii) a siRNA targeted to a transcript encoding a ps20 polypeptide; or

(iii) an antisense ps20 polynucleotide

to said subject in an amount effective to reduce or inhibit ps20 polypeptide in said subject. Suitably said method comprises administering anti-ps20 neutralising antibody to said subject. Suitably said method comprises administering monoclonal anti-ps20 antibody IG7 to said subject.

In another aspect the invention relates to a method of rendering a cell resistant to human immunodeficiency virus infection, said method comprising contacting said cell with ps20 neutralising antibody.

In another aspect the invention relates to a method of identifying an agent for inhibiting a viral infection the method comprising

(a) providing a first and a second sample comprising CD4 T cells expressing ps20;

(b) contacting said first sample with a test agent;

(c) determining level of ps20 expression in said first and second samples; and

(d) comparing the level of ps20 expression in said first and second samples;

wherein a lower level of ps20 expression in said first sample relative to said second sample identifies said test agent as an agent for inhibiting a viral infection.

In another aspect the invention relates to a method as described above further comprising the step of manufacturing a quantity of said agent so identified.

In another aspect the invention relates to a method of treating a subject, said method comprising performing the method as described above wherein said first and second samples are obtained from said subject and wherein said method further comprises administering to said subject an amount of said agent so identified.

In another aspect the invention relates to a method of rendering a cell permissive of viral infection, said method comprising inducing ps20 expression in said cell.

In another aspect the invention relates to a method of rendering a cell permissive of viral infection, said method comprising contacting said cell with a ps20 polypeptide.

Suitably said ps20 polypeptide comprises one or more of

(i) amino acids 51-65 of the ps20 sequence;

(ii) amino acids 206-220 of the ps20 sequence;

(iii) amino acids 91-105 of the ps20 sequence; or

(iv) amino acids 21-35 of the ps20 sequence.

Suitably said ps20 polypeptide comprises amino acids 21-35 of the ps20 sequence.

Suitably said ps20 polypeptide consists of one or more of

(i) amino acids 51-65 of the ps20 sequence;

(ii) amino acids 206-220 of the ps20 sequence;

(iii) amino acids 91-105 of the ps20 sequence; or

(iv) amino acids 21-35 of the ps20 sequence.

Suitably said ps20 polypeptide consists of amino acids 21-35 of the ps20 sequence.

In another aspect the invention relates to a method of inducing expression of LFA-1 in a cell, said method comprising inducing ps20 expression in said cell.

In another aspect the invention relates to a method of inducing expression of CD54 in a cell, said method comprising inducing ps20 expression in said cell.

It is important to note that cells may become infected with virus through entry of the free virus into the cell, or by spreading of the virus from cell to cell without necessarily passing through a free virus stage. These two modes by which an uninfected cell can become infected are both affected by the ps20 status of the cell. Not only are ps20 high cells more susceptible to infection by free virus, ps20 high cells are also more susceptible to cell-cell transfer of the virus. Again, this is experimentally demonstrated in the example section. In summary, if an infected cell is taken and exposed to ps20 high cells or to ps20 low cells, cell to cell transfer of the virus is more effective into ps20 high cells.

As is set out in more detail in the example section, in vitro experiments using a mixture of ps20 high and ps20 low cells exposed to virus consistently show that the virus passes more easily into ps20 high cells. These findings confirm the predictive value of assessing ps20 levels on cells, thereby experimentally validating the invention.

Numerous embodiments of the invention call for the measurement of ps20 levels. These ps20 levels are then used to make inferences about the likelihood of infection, or the infected status of the subject from which those samples were taken. ps20 is a cell surface (cell-associated) protein as well as being a secreted protein. It is demonstrated herein that the presence of elevated ps20 on the cell surface (ie. cell-associated ps20) correlates with infection. In other words, the finding of a higher level of ps20 on the cell surface makes the robust statistical inference of a greater likelihood of infection.

The secreted ps20 is also a very useful marker of infection. Typically, secreted ps20 may be determined by measuring the ps20 levels in plasma, for example, by ELISA assay. In this setting, it is also clearly experimentally demonstrated that secreted ps20 (eg, plasma ps20) is a robust statistical marker of infection.

Of course, it may be desired to measure “total” ps20. For example, this may be done by measurement of ps20 RNA levels such as mRNA levels. This also represents a robust statistical marker of infection. Indeed, in some embodiments, it may be desirable or easer to obtain nucleic acid sample in order to perform total mRNA analysis. However, more typically, assay of ps20 polypeptide level is preferred.

Peripheral T-cells represent an exemplary sample according to the present invention. These are susceptible of ps20 analysis by intracellular staining as well as cell surface staining. Such staining is easily analysed and quantified by flow cytometry. It is also possible to use extracted nucleic acid such as extracted RNA as a sample according to the present invention. This has the advantage of being easy to extract and to manipulate in vitro.

Assays

Numerous assays are described in the example section of this application. One such assay is the assay of nucleic acid such as RNA, for example to quantify the amount of ps20 transcript present in the sample, suitably with normalisation in order to obtain a value for the approximate number of transcripts per cell. This is particularly suitable when the sample comprises nucleic acid.

Intracellular staining of ps20 polypeptide may be used in order to assay ps20 levels according the present invention.

Extracellular staining of ps20 such as staining of cell surface ps20 may be used as a convenient way of quantifying ps20 levels according the present invention.

Clearly, any technique involving immunostaining of ps20 (whether intracellular or extracellular (cell surface)) may be conveniently combined with flow cytometry analysis in order to assist in data collection.

Most suitably, an ELISA-based assay is used to quantify ps20 according to the present invention. This has the advantage of being cheap to run, has the further advantage of being rapid to analyse, and is especially suitable when the sample comprises blood plasma.

Reference Sample

The reference sample may be any suitable comparable sample to that being analysed. Suitably the sample is obtained from a normal individual. Suitably the sample is obtained from an uninfected individual, ie, an individual who is known not to harbour the virus of interest.

In some embodiments, the reference sample may be comprised by a previously determined reference value, for example a particular molarity of ps20 or a particular mass of ps20 detected. However, more preferably the assays of the invention each comprise a reference sample, and ps20 levels are determined in a relative manner by comparison to the reference sample. This embodiment has the advantage of avoiding possible confounding of the results by determination of varying absolute values for the reference sample when the reference sample comprises a reference value. By always incorporating a reference sample into the assay being conducted, then a more robust and reliable indication of whether the amount of ps20 in the sample of interest is higher or lower than that found in the reference sample may be consistently obtained. For this reason, suitably the assays of the invention always comprise a reference sample determined in parallel to the sample of interest.

Therapeutic—Application to Diverse Viruses

Firstly, it should be noted that ps20 null mice have been produced. These mice are viable. This itself is an important finding. This demonstrates that it is possible to entirely remove ps20 from an animal without adversely affecting it. In Other words, ps20 is redundant for survival. This is a very strong validation of the therapeutic approaches of the invention which are aimed at neutralising or reducing ps20, since it is clearly shown that a complete absence of ps20 is no bar to the healthy survival of a subject being treated.

The invention may be applied to the treatment of a range of viruses. For example, the invention may be applied to the treatment or prevention of influenza virus infection. Furthermore, the invention may be applied to the treatment or prevention of human immunodeficiency virus. The fact that such diverse viruses can each be treated according to the same method disclosed herein, ie, by reducing or neutralising ps20, demonstrates that the invention has broad applicability to a wide range of viruses.

Therapeutic Antibodies

Any antibody raised against ps20 polypeptide may find application in the present invention. This includes polyclonal antibodies raised against the whole ps20 protein. This also includes monoclonal antibodies raised against whole ps20 polypeptide. This may also include monoclonal antibodies raised against particular epitopes or peptides taken from within the ps20 polypeptide, as is explained in more detail below.

Suitably, the antibody of the invention is one raised against one or more of the particular ps20 peptides disclosed herein. In particular, references made to example 6 and to the peptides disclosed therein. It should be noted that peptide 555 (representing amino acids 21 to 35 of ps20) represents one of the most useful epitopes against which antibodies of the invention may be raised. Thus, suitably the antibody of the invention is an antibody which reacts with a peptide comprising amino acids 21 to 35 of human ps20. A most preferred example of this is the IG7 monoclonal antibody.

It should be noted that a whole immunoglobulin has two arms. These two arms are part of the classic “Y” shape of the immunoglobulin itself. Such intact or whole immunoglobulins can work as agonists, particularly when their target is a cell surface or cell-associated protein. This can happen when each of the two arms binds to a protein on the cell, and effectively cross-links it. This cross-linking event can send a positive signal instead of blocking or neutralising the target polypeptide. Numerous antibodies in the art are known to suffer from this drawback. For this reason, preferably the antibody of the invention is a single chain immunoglobulin (sc) molecule. In other words, most suitably the antibody may be a single chain antibody rather than a bivalent immunoglobulin molecule. Using a single chain antibody has the advantage of being able to sequester or neutralise all of the target antigen present, yet without suffering the drawback of cross-linking those molecules. Most suitably, the antibody of the invention is a single chain antibody having the specificity to recognise a polypeptide comprising amino acids 21 to 35 of ps20, ideally a single chain antibody having the specificity of IG7. In case any guidance is needed, it should be noted that the making of a single chain antibody is an entirely standard and routine procedure for the person skilled in the art. Most typically, single chain antibodies are derived by a simple papain digestion of the whole antibody molecule. Thus, a preferred single chain antibody of the invention is obtained by the digestion of IG7 antibody by the action of papain.

IG7 antibody is available via Baylor College of Medicine, USA.

Of course, it is possible to make synthetic antibodies. Ideally, when the antibody of the invention is a synthetic antibody, said antibody would have the variable region sequences of the IG7 monoclonal antibody. Of course, it is a straightforward matter to determine the amino acid sequence of that antibody, and then to manufacture the synthetic antibody using any of the numerous commercially available synthesising services.

Suitably the antibody is a single chain antibody recognising any of the ps20 peptides shown in the accompanying examples.

Suitably the antibody of the invention neutralises ps20. In case any further guidance is needed, candidate antibodies may be easily tested as to whether or not they are neutralising by following the procedures set out in example 6 below, in particular by following the HIV infection assay in the presence or absence of the particular antibody of interest.

Other entities may be used to make a therapeutic intervention according to the present invention. Examples include short interfering RNA (siRNA) targeted against ps20; antisense nucleic acids against ps20; or lentiviral delivery of short hairpin nucleic acids to knock down the ps20 transcript. In principle, any technique for reducing or suppressing ps20 expression, or any reagent capable of neutralising ps20 polypeptide is a useful therapeutic tool for application in the present invention.

Ps20 polypeptides and polynucleotides encoding the ps20 polypeptides, have been found to have application in the determination of the susceptibility of CD4 T cells to viral infection, as well as for prognosis and treatment of viral diseases.

Ps20 polypeptides including native-sequence polypeptides, isoforms, chimeric polypeptides, all homologs, fragments, and precursors of the polypeptides, as well as modified forms of the polypeptides and derivatives, are referred to herein as “ps20 polypeptide(s)”. Polynucleotides encoding ps20 polypeptides are referred to herein as “ps20 polynucleotide(s)” or “polynucleotides encoding ps20 polypeptide(s)”. The ps20 polypeptides and ps20 polynucleotides are sometimes collectively referred to herein as “ps20 marker(s)”.

The invention relates to therapeutic applications for viral diseases employing ps20 polypeptides, ps20 polynucleotides, and/or modulators of ps20 polypeptides and/or ps20 polynucleotides.

In an aspect, the invention relates to compositions comprising ps20 polypeptides or parts thereof associated with a viral disease, or modulators of ps20 polypeptides associated with a viral disease, and a pharmaceutically acceptable carrier, excipient, or diluent. In an embodiment, the invention relates to compositions comprising antagonists of ps20 polypeptides or ps20 polynucleotides associated with a viral disease, and a pharmaceutically acceptable carrier, excipient or diluent. Thus in another embodiment the invention relates to a composition comprising antibody capable of binding ps20 polypeptide, siRNA targeted to a transcript encoding a ps20 polypeptide, or antisense ps20 polynucleotide and a pharmaceutically acceptable carrier, excipient, or diluent.

A method for treating or preventing a viral disease in a subject is also provided comprising administering to a subject in need thereof modulators of ps20 polypeptides or parts thereof associated with a viral disease. In an aspect, a method is providing for treating or preventing a viral disease in a subject comprising administering to a subject in need thereof, an antagonist of a ps20 polypeptide or a ps20 polynucleotide, or a composition of the invention. In an aspect the invention provides a method of treating a subject afflicted with or at risk of developing a viral disease comprising inhibiting expression of ps20 polypeptides. In an embodiment, the invention provides a method of treating a subject afflicted with or at risk of developing, a viral disease comprising inhibiting expression of ps20 polypeptides by administering an antisense ps20 polynucleotide or an interfering RNA (siRNA) targeted to a transcript encoding a ps20 polypeptide.

In an aspect, the invention provides binding agents, in particular therapeutic antibodies, specific for ps20 polypeptides associated with a viral disease that can be used to destroy or inhibit the disease (e.g. the growth of the virus or destruction or rescue of selected CD4 T cells that are susceptible to viral infection), or to block ps20 polypeptide activity associated with a disease. In an aspect, ps20 polypeptides may be used in various immunotherapeutic methods to promote immune-mediated destruction or growth inhibition of CD4 T cells secreting ps20 polypeptides.

The invention also contemplates a method of using ps20 polypeptides or parts thereof, or modulators of ps20 polypeptides, in the preparation or manufacture of a medicament for the prevention or treatment of a viral disease.

Another aspect of the invention is the use of ps20 polypeptides, peptides derived therefrom, or chemically produced (synthetic) peptides, or any combination of these molecules, for use in the preparation of vaccines to prevent a viral disease and/or to treat a viral disease. Therefore, the invention contemplates vaccines for stimulating or enhancing in a subject to whom the vaccine is administered production of antibodies directed against one or more ps20 polypeptides.

The invention provides an immunogenic composition for protecting subjects against a viral infection. An immunogenic composition of the invention comprises an immunogenic amount of a region of a ps20 polypeptide. In a composition of the invention, the region of a ps20 polypeptide defines an epitope which induces, the formation of antibodies against the virus. In aspects of the invention the region of a ps20 polypeptide is immunoreactive and found in selected viruses, for example, HIV.

In embodiments of the invention an immunogenic composition comprises synthetic peptides about 5 to 100, 5 to 200, 10 to 150, 10 to 100, 20 to 100, 10 to 50 or 20 to 25 amino acids in length which are portions of a ps20 polypeptide. In embodiments, the synthetic peptides are serotype specific peptides. Synthetic peptides may be used, for example, individually, in a mixture, or in a polypeptide or protein. For example, a polypeptide or protein can be created by fusing or linking the peptides to each other, synthesizing the polypeptide or protein based on the peptide sequences, and linking or fusing the peptides to a backbone. In addition, a liposome may be prepared with the peptides conjugated to it or integrated within it.

The invention also provides a method for stimulating or enhancing in subject production of antibodies directed against one or more ps20 polypeptide. The method comprises administering to the subject an immunogenic composition or vaccine of the invention in a dose effective for stimulating or enhancing production of the antibodies.

The invention further provides a method for treating, preventing, or delaying recurrence of a viral disease. The method comprises administering to the subject a vaccine of the invention in a dose effective for treating, preventing, or delaying recurrence of a viral disease.

The invention also provides a method for assessing the potential efficacy of a test agent for inhibiting a viral disease, and a method of selecting an agent for inhibiting a viral disease.

Other objects, features and advantages of the present invention will become apparent from the following detailed description. It should be understood, however, that the detailed description and the specific examples while indicating preferred embodiments of the invention are given by way of illustration only, since various changes and modifications, within the spirit and scope of the invention will become apparent to those skilled in the art from this detailed description.

### DESCRIPTION OF THE DRAWINGS

The invention will now be described in relation to the drawings in which:

FIG. 1 is a graph showing polyclonal antibody binding to ps20 mRNA high G91, Jurkats compared to ps20 mRNA low EV control.

FIG. 2 are graphs showing that rabbit polyclonal antibody blocks HIV infection of cells that express endogenous ps20 (EV2) but has no effect on a ps20 negative cell (H9).

FIG. 3 shows graphs and bar charts showing HIV infection is suppressed by ps20 knockdown: siRNA-mediated knockdown of endogenous ps20 blocks HIV infection (a) 2×10⁵ HeLa indicator cells were exposed to transfection reagent in absence of siRNA (mock) or 50 nM siRNA specific for ps20 or MAPK. 48 hours later, adherent cells were harvested by trypsinisation, washed and viable cells reseeded at a density of 2×10⁴ cells per well and left to adhere for 6 hours before addition of virus (5 ul, 25 ul, 125 ul). 36 hours later productive HIV infection was determined in cell lysates using β-galactosidase levels measured as relative light units (RLU) (minus background RLU by uninfected cells) in a luminometer. (b)/(c) Parallel cultures as above were set-up and samples processed for ps20 mRNA or MAPK mRNA by qRT-PCR. Non-specific effect of MAPK siRNA on ps20-knockdown is shown in 3b and vice versa of ps20 siRNA on MAPK in 6c. Error bars represent mean of three replicates.

FIG. 4 shows graphs and plots showing that anti-ps20 monoclonal antibody IG7 suppresses HIV spread in in vitro CD4+ T cell cultures and more specifically that anti-ps20 Ab IG7inhibits spread of X4 and R5 HIV-1 strains in diverse CD4 T cell populations.

FIG. 5 shows graphs illustrating that anti-ps20 rabbit polyclonal antibody suppresses HIV spread in vitro CD4+ T cell cultures; in more detail, rabbit polyclonal anti-ps20 antibody blocks HIVinfection of cells that express endogenous ps20 (EV2) but has no effect on a ps20 negative cell (H9).

FIG. 6 shows graphs and bar charts of siRNA-mediated knockdown of endogenous ps20 suppresses HIV spread.

FIG. 7 shows bar charts showing that ps20 promotes T-T cell-cell transfer of HIV in primary CD4 T lymphocytes and Anti-ps20 antibody blocks virus transfer

FIG. 8 shows graphs and bar charts showing exogenous addition of ps20 or stable endogenous ps20 expression by retroviral transduction promotes HIV infection

FIG. 9 shows graphs of a number of peptides that mimic the HIV enhancing effect of recombinant Ps20 are identified; ps20 peptides mimic recombinant protein effect in CEM G 37 CD4 T-cells Peptide 555 appears to be the most potent with the potentiating effect titrating down to 3 ug/ml peptide. Peptide doses tested=30, 3,0.3 and 0.03 ug/ml.

FIG. 10 shows graphs of 555 ps20 peptide potently mimics the HIV enhancing effect of recombinant Ps20 Dose Range tested: 20-0.2 ug/ml Effective: 13.3 uM (upto ×20 fold enhancement) 2.7 uM (upto ×4 fold enhancement)

FIG. 11 shows plots illustrating 555 ps20 peptide enhances cell-cell HIV transfer in primary CD4 T cells

FIG. 12 shows bar charts of evidence for immunomodulatory role of ps20. ps20 enhances HIV infection by up-regulating cell surface cell adhesion antigen CD54, which is of known importance in promoting HIV infection.

FIG. 13 shows a bar chart showing that ps20 is a broad spectrum anti-viral target. Endogenous ps20 encoded by the murine wfdc1 gene is a permissivity factor for influenza virus infection in C57B16 mice. The wfdc1 −/− mouse survives, breeds normally under pathogen-free conditions. Virus: Influenza A/Tx strain Titer: 10 TCID50 as determined by infection of Mason Darby Canine Kidney (MDCK) cells. Challenge: 10,100,1000 TCID50. Only lower dose showed difference (wt and het mice have 2-3 logs higher virus titer than null). Virus titer: Lung extracts titred on MDCK. Virus titers are higher in the presence of ps20.

FIG. 14 shows bar charts of influenza results.

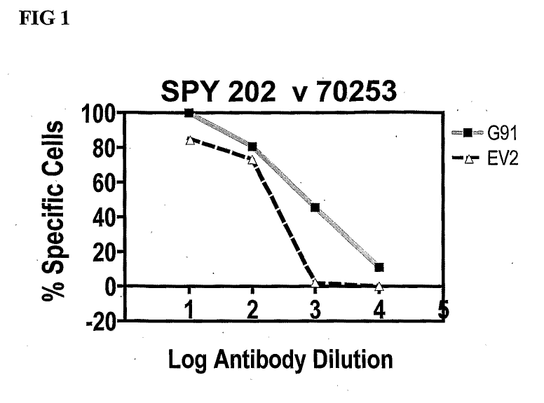

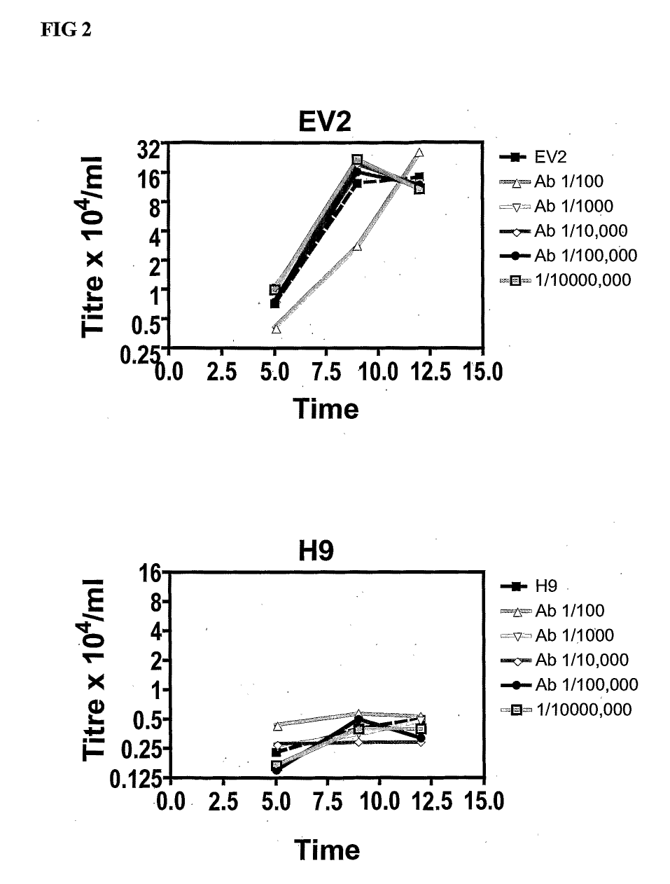

### DETAILED DESCRIPTION OF THE INVENTION

Methods are provided for assessing the efficacy of one or more test agents for inhibiting a viral disease, assessing the efficacy of a therapy for a viral disease, selecting an agent or therapy for inhibiting a viral disease, treating a patient afflicted with a viral disease, inhibiting a viral disease in a patient, and assessing the disease potential of a test agent.

### Glossary

In accordance with the present invention there may be employed conventional biochemistry, enzymology, molecular biology, microbiology, and recombinant DNA techniques within the skill of the art. Such techniques are explained fully in the literature. See for example, Sambrook et al, Molecular Cloning: A Laboratory Manual, Third Edition (2001) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. Thus, for example, reference to a composition containing “a modulator” includes a mixture of two or more modulators. The term “about” means plus or minus 0.1 to 50%, 5-50%, or 10-40%,. preferably 10-20%, more preferably 10% or 15%, of the number to which reference is being made.

The terms “peptide”, “polypeptide” and “protein” are used interchangeably and as used herein refer to more than one amino acid joined by a peptide bond.

The term “effective amount” or “effective dose” refers to a non-toxic but sufficient amount of an agent (e.g. antibody) to provide the desired biological effect. The exact amount required will vary from subject to subject, depending on the species, age and general condition of the subject, the particular agent used, its mode of administration, and the like. An appropriate effective amount or effective dose may be determined by one or ordinary skill in the art using routine experimentation.

“Pharmaceutically acceptable” refers to a material that is not biologically or otherwise undesirable, i.e., the material may be administered to an individual without causing any undesirable biological effects or interacting in a deleterious manner with any of the other components of a composition in which it is contained.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. The use of such media and agents for an active substance is well known in the art.

“Synthetic” refers to items, e.g., peptides, which are not naturally occurring, in that they are isolated, synthesized or otherwise manipulated by man.

“Immunogenic” as used herein encompasses materials which are capable of producing an immune response.

“Composition” includes any composition of matter, including peptides, polypeptides, proteins, mixtures, vaccines, antibodies, or markers of the present invention.

“Viral diseases” means a class of diverse diseases and disorders caused by or believed to be caused by viruses. The term includes any stage of a viral infection, including incubation phase, latent or dormant phase, acute phase, and development and maintenance of immunity towards a virus. Consequently, the term “treatment' is meant to include aspects of generating or restoring immunity of the patient's immune system, as well as aspects of suppressing or inhibiting virus activity. “Virus activity” includes virus replication, assembly, maturation, envelopment, extracellular virus formation, virus egress, and virus transmission. Viral diseases include, without limitation, genital warts (HPV), HIV/AIDS, herpes, influenza, measles, polio, varicella-zoster, hepatitis A, hepatitis B, hepatitis C, hepatitis D, herpes simplex virus (type 1 and type 2), hepatitis E, hepatitis G, cytomegalovirus, meningitis, genital warts (HPV), a disease associated with respiratory syncytial virus infection, a disease associated with coxsackie virus infection, a disease associated with ebola virus infection, a disease associated with hantavirus infection, a disease associated with human papilloma virus infection, a disease associated with rotavirus infection, a disease associated with west nile virus infection, a disease associated with Epstein-Barr virus infection, a disease associated with papilloma virus infection, a disease associated with influenza virus infection, vesticular stomatitis virus infection, and dengue fever. The clinical sequelae of viral infections include without limitation, herpes, AIDS, lassa fever, kaposi's sarcoma, meningitis, mumps, polio, chicken pox, colds and flu, dengue fever, encephalitis, Fifth disease, shingles, genital warts, rubella, yellow fever, hepatitis A, B and C, measles, rabies, and smallpox. The singular form “viral disease” includes any one or more diseases selected from the class of viral diseases, and includes any compound or complex disease state wherein a component of the disease state includes a disease selected from the class of viral diseases.

The terms “subject” or “patient” refer to an animal including a warm-blooded animal such as a mammal, which is afflicted with or suspected of having or being pre-disposed to a condition or disease described herein. Mammal includes without limitation any members of the Mammalia. In general, the terms refer to a human. The terms also include animals bred for food, sport, or as pets, including domestic animals such as horses, cows, sheep, poultry, fish, pigs, and goats, and cats, dogs, and zoo animals, apes (e.g. gorilla or chimpanzee), and rodents such as rats and mice. The methods herein for use on subjects contemplate prophylactic as well as curative use. Typical subjects for treatment include persons susceptible to, suffering from or that have suffered a viral disease.

The term “ps20 polypeptide” includes human ps20, in particular the native-sequence polypeptide, isoforms, chimeric polypeptides, all homologs, fragments, precursors, complexes, and modified forms and derivatives of human ps20. The amino acid sequence for native human ps20 includes the sequences of Accession Nos. NP_{—}067020, EAW95486, EAW95487, AAG16647, AAG15263.1, Q9HC57, BAC11377.1, ABM84291.1, or ABM87681.1 or shown in SEQ ID NO. 2 and 3.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having substantial sequence identity. In an aspect a polypeptide variant has at least about 45%, preferably at least about 85%, more preferably at least about 90%, most preferably at least about 95% amino acid sequence identity with a native-sequence polypeptide. Polypeptide variants preferably retain the immunogenic activity of a corresponding native-sequence polypeptide. Particular polypeptide variants have at least 45%, preferably 50%, 55%, 60%, 65%, 70%, 75%, 80%, 85%, 90%, 95%, 97%, 98%, or 99% sequence identity to the sequences identified in Accession Nos. NP_{—}067020, EAW95486, EAW95487, AAG16647, AAG15263.1, Q9HC57, BAC11377.1, ABM84291.1, or ABM87681.1, or shown in SEQ ID NO. 2 and 3. Polypeptide variants also include, for instance, polypeptides wherein one or more amino acid residues are added to, or deleted from, the N- or C-terminus of the full-length or mature sequences of the polypeptide, including variants from other species, but excludes a native-sequence polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et at, Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S., et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

A variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine polypeptide.

Ps20 polypeptides include chimeric or fusion proteins. A “chimeric protein” or “fusion protein” comprises all or part (preferably biologically active) of ps20 polypeptide operably linked to a heterologous polypeptide (i.e., a polypeptide other than a ps20 polypeptide). Within the fusion protein, the term “operably linked” is intended to indicate that a ps20 polypeptide and the heterologous polypeptide are, fused in-frame to each other. The heterologous polypeptide can be fused to the N-terminus or C-terminus of a ps20 polypeptide. A useful fusion protein is a GST fusion protein in which a ps20 polypeptide is fused to the C-terminus of GST sequences. Another example of a fusion protein is an immunoglobulin fusion protein in which all or part of a ps20 polypeptide is fused to sequences derived from a member of the immunoglobulin protein family. Chimeric and fusion proteins can be produced by standard recombinant DNA techniques.

A modified form of a polypeptide referenced herein includes modified forms of the polypeptides and derivatives of the polypeptides, including but not limited to glycosylated, phosphorylated, acetylated, methylated or lapidated forms of the polypeptides. For example, an N-terminal methionine may be cleaved from a to polypeptide, and a new N-terminal residue may or may not be acetylated.

Ps20 polypeptides may be prepared by recombinant or synthetic methods, or isolated from a variety of sources, or by any combination of these and similar techniques.

“Ps20 polynucleotide(s)” refers to polynucleotides encoding ps20 polypeptides including native-sequence polypeptides, polypeptide variants including a portion of a polypeptide, an isoform, precursor, complex, a chimeric polypeptide, or modified forms and derivatives of the polypeptides. A polynucleotide encoding a native polypeptide employed in the present invention includes the polynucleotides encoding ps20 [e.g., Accession Nos. NM_{—}021197, AF169631, AAG16647.1, AF302109, AAG15263.1, AK075061, BAC11377.1, BCO29159, AAH29159.1, AC010551.3, CH471114.2, AL713785, AL713785, CR595501, CR604862, CR608359, CR610530, CR615719, DQ893365.2, or DQ896682.2, Gene ID No. 58189, or SEQ ID NO. 1].

Ps20 polynucleotides include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

Ps20 polynucleotide further include sequences that differ from a native sequence [e.g. Accession Nos. NM_{—}021197, AF169631, AAG16647.1, AF302109, AAG15263.1, AK075061, BAC11377.1, BC029159, AAH29159.1, AC010551.3, CH471114.2, AL713785, AL713785, CR595501, CR604862, CR608359, CR610530, CR615719, DQ893365.2, or DQ896682.2, Gene ID No. 58189, or SEQ ID NO. 1] due to degeneracy in the genetic code. As one example, DNA sequence polymorphisms within the nucleotide sequence of a ps20 polypeptide may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Ps20 polynucleotides also include nucleic acids that hybridize under stringent conditions, preferably high stringency conditions to a ps20 polynucleotide [e.g. Accession Nos. NM_{—}021197, AF169631, AAG16647.1, AF302109, AAG15263.1, AK075061, BAC11377.1, BC029159, AAH29159.1, AC010551.3, CH471114.2, AL713785, AL713785, CR595501, CR604862, CR608359, CR610530, CR615719, DQ893365.2, or DQ896682.2, Gene ID No. 58189, or SEQ ID NO. 1]. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Ps20 polynucleotides also include truncated nucleic acids or nucleic acid fragments and variant forms of the nucleic acids that arise by alternative splicing of an mRNA corresponding to a DNA.

The ps20 polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods bf the invention may be of any length suitable for a particular method. In certain applications the term refers to antisense polynucleotides (e.g. mRNA or DNA strand in the reverse orientation to sense ps20 polynucleotides).

A “significant difference” in levels of ps20 polypeptides or polypeptides in a sample compared to a control or standard may represent levels that are higher or lower than the standard error of the detection assay. In particular embodiments, the levels may be 1.5, 2, 3, 4, 5, or 6 times higher or lower than the control or standard.

“Binding agent” refers to a substance that specifically binds to one or more ps20 polypeptides. A substance “specifically binds” to one or more ps20 polypeptides if is reacts at a detectable level with one or more ps20 polypeptides, and does not react detectably with peptides containing an unrelated or different sequence. Binding properties may be assessed using an ELISA, which may be readily performed by those skilled in the art (see for example, Newton et al., Develop. Dynamics 197: 1-13, 1993).

A binding agent may be a ribosome, with or without a peptide component, an aptamer, an RNA molecule, or a polypeptide. A binding agent may be a polypeptide that comprises one or more ps20 polypeptide sequence, a peptide variant thereof, or a non-peptide mimetic of such a sequence. By way of example, a ps20 polypeptide sequence may be a peptide portion of a ps20 polypeptide that is capable of modulating a function mediated by a ps20 polypeptide.

An aptamer includes a DNA or RNA molecule that binds to nucleic acids and proteins. An aptamer that binds to a protein (or binding domain) of a ps20 polypeptide or a ps20 polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies include, but are not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to a ps20 polypeptide, one or more complementarity determining regions (CDRs) of an anti-ps20 polypeptide antibody). Preferably agonistic antibodies or fragments thereof that immunospecifically bind to a ps20 polypeptide or fragment thereof preferentially agonize a ps20 polypeptide and do not significantly agonize other activities.

An antibody of the present invention also includes immunoglobulin types IgA, IgD, IgE, IgG, IgM and subtypes of any of the foregoing, wherein the light chains of the immunoglobulin may be kappa or lambda type.

Antibodies may be monospecific, bispecific, trispecific or of greater multispecificity. Multispecific antibodies may immunospecifically bind to different epitopes of a ps20 polypeptide or may immunospecifically bind to both a ps20 polypeptide as well as a heterologous epitope, such as a heterologous polypeptide or solid support material.

In aspects of the invention, the antibodies immunospecifically bind to ps20 polypeptides including fragments thereof Antibodies that immunospecifically bind to ps20 polypeptides include antibodies or fragments thereof that specifically bind to a ps20 polypeptide or a fragment of a ps20 polypeptide and do not specifically bind to other non-ps20 polypeptides. In embodiments of the invention, antibodies that immunospecifically bind to a ps20 polypeptide or fragment thereof do not non-specifically cross-react with other antigens (e.g., binding cannot be competed away with a non-ps20 polypeptide). Antibodies or fragments that immunospecifically bind to a ps20 polypeptide can, be identified, for example, by immunoassays or other techniques known to those of skill in the art.

Antibodies may be from any animal origin including birds and mammals (e.g., human, murine, donkey, sheep, rabbit, goat, guinea pig, camel, horse, or chicken). In aspects of the invention, the antibodies are human or humanized monoclonal antibodies.

In aspects of the invention, the antibody is a humanized antibody. A “humanized antibody” includes forms of non-human antibodies that are chimeric antibodies which comprise minimal sequence derived from non-human immunoglobulin. Humanized antibodies may be human immunoglobulins (recipient antibody) in which hypervariable region residues of a recipient are replaced by hypervariable region residues from a non-human species (donor antibody) such as mouse, rat, rabbit or non-human primate having the desired specificity, affinity, and capacity. In some cases, Framework Region (FR) residues of the human immunoglobulin are replaced by corresponding non-human residues. In addition, humanized antibodies may comprise residues which are not found in the recipient antibody or in the donor antibody, for example, modifications to further refine antibody performance. A humanized antibody will typically comprise substantially all of at least one, and typically two, variable domains, in which all or substantially all of the hypervariable regions correspond to those of a non-human immunoglobulin and all or substantially all of the Framework Regions are those of a human immunoglobulin sequence. A humanized antibody may optionally comprise at least a portion of an immunoglobulin constant region (Fc), typically that of a human immunoglobulin that immunospecifically binds to a ps20 polypeptide that has been modified by the introduction of amino acid residue substitutions, deletions or additions (i.e., mutations). In aspects of the invention, a humanized antibody is a derivative that comprises amino acid residue substitutions, deletions or additions in one or more non-human CDRs. A derivative may have substantially the same binding, better binding, or poorer binding when compared to a non-derivative humanized antibody. [See the following for details of humanized antibodies: U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-25; Caldas et al., 2000, Protein Eng. 13:353-60; Morea et al., 2000, Methods 20:267-79; Baca et al., 1997, J. Biol. Chem. 272:10678-84; Roguska et al., 1996, Protein Eng. 9:895-904; Couto et al., 1995, Cancer Res. 55 (23 Supp):5973s.sup.-5977s; Couto et al., 1995, Cancer Res. 55:1717-22; Sandhu, 1994, Gene 150:409-10; Pedersen et al., 1994, J. Mol. Biol. 235:959-73; Jones et al., 1986, Nature 321:522-525; Reichmann et al., 1988, Nature 332:323-329; and Presta, 1992, Curr. Op. Struct. Biol. 2:593-596.

Antibodies may be prepared using methods known to those skilled in the art. Isolated native or recombinant ps20 polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for ps20 polypeptides may also be obtained from scientific or commercial sources. In an embodiment of the invention, antibodies are reactive against ps20 polypeptides if they bind with a Kₐ of greater than or equal to 10⁻⁷ M.

In aspects of the invention, the antibody is a purified antibody. By “purified” is meant that a given antibody or fragment thereof, whether one that has been removed from nature (isolated from blood serum) or synthesized (produced by recombinant means), has been increased in purity, wherein “purity” is a relative term, not “absolute purity.” In particular aspects, a purified antibody is 60% free, preferably at least 75% free, and more preferably at least 90% free from other components with which it is naturally associated or associated following synthesis.

### Methods for Identifying or Evaluating Substances/Compounds

The invention contemplates methods designed to identify substances that modulate the biological activity of a ps20 polypeptide including substances that bind to a ps20 polypeptide or portion thereof, or bind to other proteins that interact with a ps20 polypeptide, to compounds that interfere with, or enhance the interaction of a ps20 polypeptide and substances that bind to a ps20 polypeptide, or other proteins that interact with a ps20 polypeptide. Methods can also be utilized that identify compounds that bind to regulatory sequences of a ps20 polynucleotide.

Substances, agents and compounds that may be identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], polynucleotides (e.g., siRNA) and small organic or inorganic molecules. The substance, agent or compound may be an endogenous physiological compound or it may be a natural or synthetic compound.

Substances identified using the methods of the invention may be isolated, cloned and sequenced using conventional techniques. A substance that associates with a ps20 polypeptide of the invention may be an agonist or antagonist of the biological or immunological activity of the polypeptide. The term “agonist”, refers to a molecule that increases the amount of, or prolongs the duration of, the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention.

Substances which modulate a ps20 polypeptide can be identified based on their ability to bind to a ps20 polypeptide. Therefore, the invention also provides methods for identifying substances which bind to a ps20 polypeptide. Substances which can bind with a ps20 polypeptide may be identified by reacting a ps20 polypeptide with a test substance which potentially binds to a ps20 polypeptide, under conditions which permit the formation of substance-ps20 polypeptide complexes and removing and/or detecting the complexes. The complexes can be detected by assaying for substance-ps20 polypeptide complexes, for free substance, or for non-complexed ps20 polypeptide. Conditions which permit the formation of substance-ps20 polypeptide complexes may be selected having regard to factors such as the nature and amounts of the substance and the polypeptide. The substance-protein complex, free substance or non-complexed polypeptides may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against ps20 polypeptide or the substance, or labelled ps20 polypeptide, or a labelled substance may be utilized. The antibodies, polypeptides, or substances may be labelled with a detectable substance as described above.

A ps20 polypeptide, or the substance used in these methods of the invention may be insolubilized. For example, a ps20 polypeptide, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized polypeptide or substance may be prepared by reacting the material with a suitable insoluble carrier using known chemical or physical methods, for example, cyanogen bromide coupling.

The invention also contemplates a method for evaluating a compound for its ability to modulate the biological activity of a ps20 polypeptide by assaying for an agonist or antagonist (i.e., enhancer or inhibitor) of the binding of a ps20 polypeptide with a substance which binds with a ps20 polypeptide. The basic method for evaluating if a compound is an agonist or antagonist of the binding of a ps20 polypeptide and a substance that binds to the polypeptide is to prepare a reaction mixture containing the ps20 polypeptide and the substance under conditions which permit the formation of substance—ps20 polypeptide complexes, in the presence of a test compound. The test compound may be initially added to the mixture, or may be added subsequent to the addition of the ps20 polypeptide and substance. Control reaction mixtures without the test compound or with a placebo are also prepared. The formation of complexes is detected, and the formation of complexes in the control reaction but not in the reaction mixture indicates that the test compound interferes with the interaction of the ps20 polypeptide and substance. The reactions may be carried out in the liquid phase or the ps20 polypeptide, substance, or test compound may be immobilized as described herein. The ability of a compound to modulate the biological activity of a ps20 polypeptide may be tested by determining the biological effects on cells.

It will be understood that the agonists and antagonists, i.e., inhibitors and enhancers, that can be assayed using the methods of the invention may act on one or more of the binding sites on the polypeptide or substance including agonist binding sites, competitive antagonist binding sites, non-competitive antagonist binding sites or allosteric sites.

The invention also makes it possible to screen for antagonists that inhibit the effects of an agonist of the interaction of ps20 polypeptide with a substance which is capable of binding to the ps20 polypeptide. Thus, the invention may be used to assay for a compound that competes for the same binding site of a ps20 polypeptide.

The invention also contemplates methods for identifying compounds that bind to proteins that interact with a ps20 polypeptide. Protein-protein interactions may be identified using conventional methods such as co-immunoprecipitation, crosslinking and co-purification through gradients or chromatographic columns. Methods may also be employed that result in the simultaneous identification of genes which encode proteins interacting with a ps20 polypeptide. These methods include probing expression libraries with labelled ps20 polypeptide.

Two-hybrid systems may also be used to detect protein interactions in vivo. Generally, plasmids are constructed that encode two hybrid proteins. A first hybrid protein consists of the DNA-binding domain of a transcription activator protein fused to a ps20 polypeptide, and the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins may be used in the methods described herein. In particular, ps20 polypeptides fused to a glutathione-S-transferase may be used in the methods.

A modulator of a ps20 polypeptide of the invention may also be identified based on its ability to inhibit or enhance activity of the polypeptide. In aspects of the invention, substances that modulate ps20 polypeptides can be selected by assaying for a substance that inhibits or stimulates, preferably inhibits, the activity of a ps20 polypeptide. Such a substance can be identified based on its ability to specifically interfere with or stimulate, preferably interfere with, the activity of a ps20 polypeptide.

The invention also contemplates methods for evaluating test agents or compounds for their ability to reduce or inhibit viral infection or disease. Therefore, the invention provides a method for assessing the potential efficacy of a test agent for reducing or inhibiting a viral infection in a patient, the method comprising comparing:
(a) levels of one or more ps20 polypeptides, and/or ps20 polynucleotides in a sample obtained from a patient and exposed to the test agent; and
(b) levels of one or more ps20 polypeptides, and/or ps20 polynucleotides in a second sample obtained from the patient, wherein the sample is not exposed to the test agent, wherein a significant difference in the levels of expression of one or more ps20 polypeptides, and/or ps20 polynucleotides relative to the second sample, is an indication that the test agent is potentially efficacious for reducing or inhibiting a viral infection in the patient.

The first and second samples may be portions of a single sample obtained from a patient or portions of pooled samples obtained from a patient.

In an aspect, the invention provides a method of selecting an agent for inhibiting a viral disease in a patient comprising:
(a) obtaining a sample from the patient;
(b) separately maintaining aliquots of the sample in the presence of a plurality of test agents;
(c) comparing one or more ps20 polypeptides, and/or ps20 polynucleotides in each of the aliquots; and
(d) selecting one of the test agents which alters the levels of one or more. ps20 polypeptides, and/or ps20 polynucleotides relative to other test agents.

Still another aspect of the present invention provides a method of conducting a drug discovery business comprising:
(a) providing one or more methods or assay systems for identifying agents that reduce or inhibit a viral infection in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to a viral disease (e.g. HIV or influenza) comprising:
(a) maintaining separate aliquots of cells (e.g. CD4 T cells) from a patient with a viral disease in the presence and absence of the test compound; and
(b) comparing one or more ps20 polypeptides, and/or ps20 polynucleotides in each of the aliquots.

A significant difference between the levels of the markers in the aliquot maintained in the presence of (or exposed to) the test compound relative to the aliquot maintained in the absence of the test compound, indicates that the test compound possesses the potential to contribute to a viral disease.

### Therapeutic Applications

Ps20 antagonists, in particular antibodies, ps20 polynucleotides and substances, agents, or compounds identified by the methods described herein, may be used for modulating the biological activity of a ps20 polypeptide, and they may be used in the treatment of viral diseases. The ps20 markers may be involved in processes that modulate virus activity, and in aspects of methods of treatment or prevention disclosed herein virus activity may be reduced or inhibited.

Accordingly, ps20 antagonists may be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to living organisms including humans and animals. Administration of a therapeutically active amount of a pharmaceutical composition of the present invention is defined as an amount effective, at dosages and for periods of time necessary to achieve the desired result. For example, a therapeutically active amount of a substance may vary according to factors such as the disease state, age, and weight of the individual, and the ability to elicit a desired response in the individual. Dosage regima may be adjusted to provide the optimum therapeutic response. For example, several divided doses may be administered daily or the dose may be proportionally reduced as indicated by the exigencies of the therapeutic situation.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington: The Science and Practice of Pharmacy. (21st Edition, Popovich, N (eds), Advanced Concepts Institute, University of the Sciences in Philadelphia, Philadelphia, Pa. 2005). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies.

In aspects of the invention, methods are provided for reducing or inhibiting a viral infection comprising directly or indirectly inhibiting a ps20 polypeptide, preferably inhibiting a ps20 polypeptide of SEQ ID NO. 2 or 3. In an embodiment of the invention, a method is provided for reducing or inhibiting a viral infection in a subject comprising, administering an effective amount of a substance which is an inhibitor of a ps20 polypeptide. In particular, methods are provided for treating a patient suffering from or who may be susceptible to a viral disease.

In an embodiment of the invention a method is provided for treating a patient or who may be susceptible to a viral disease (e.g., HIV) comprising administering therapeutically effective dosages of an inhibitor identified in accordance with a method of the invention or described herein. Treatment with the inhibitor is discontinued after ps20 polypeptide levels are within normal range, and before any adverse effects of administration of the inhibitor are observed.

A substance that reduces or inhibits a viral infection may be a molecule which interferes with the transcription and/or translation of a ps20 polypeptide, in particular a ps20 polypeptide of SEQ ID NO. 1. For example, the sequence of a nucleic acid molecule encoding a ps20 polypeptide or fragments thereof may be inverted relative to its normal presentation for transcription to produce an antisense nucleic acid molecule. An antisense nucleic acid molecule may be constructed using chemical synthesis and enzymatic ligation reactions using procedures known in the art.

Genes encoding a ps20 polypeptide can be turned off by transfecting a cell or tissue with vectors which express high levels of a desired ps20 polypeptide-encoding fragment. Such constructs can inundate cells with untranslatable sense or antisense sequences. Even in the absence of integration into the DNA, such vectors may continue to transcribe RNA molecules until all copies are disabled by endogenous nucleases. Vectors can be derived from retroviruses, adenovirus, herpes or vaccinia viruses, or from various bacterial plasmids, and used to deliver polynucleotides to a targeted cell population. Antisense sequences may also be introduced using lipid-based transfection technologies

Modifications of gene expression can be obtained by designing antisense molecules, DNA, RNA or PNA, to the regulatory regions of a gene encoding a polypeptide of the invention, i.e., the promoters, enhancers, and introns. Preferably, oligonucleotides are derived from the transcription initiation site, for example, between about −10 and +10 regions of the leader sequence. Antisense molecules may also be designed so that they block translation of mRNA by preventing the transcript from binding to ribosomes (i.e., micRNA). Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Can (1994) Molecular and Immunologic Approaches, Futura Publishing Co, Mt Kisco N.Y.). Methods well known to those skilled in the art may be used to construct recombinant vectors which will express antisense ps20 polynucleotides. (See, for example, the techniques described in Sambrook et al (supra) and Ausubel et al (supra)).

The invention provides a method of inhibiting expression of a gene encoding a ps20 polypeptide comprising the step of (i) providing a biological system in which expression of a gene encoding a ps20 polypeptide is to be inhibited; and (ii) contacting the system with an antisense molecule that hybridizes to a transcript encoding a ps20 polypeptide.

Antisense RNA transcripts of the present invention can have a base sequence complementary to part or all of a ps20 polynucleotide and modulate expression of ps20 polynucleotides. Antisense nucleic acids are generally single-stranded nucleic acids (DNA, RNA, modified DNA, or modified RNA) complementary to a portion of a target nucleic acid (e.g., an mRNA ps20 polynucleotide transcript) and are able to bind to the target to form a duplex. In aspects of the invention, an antisense is an oligonucleotide ranging from 10 to 50, in particular 15 to 35 nucleotides in length. Binding of the antisense molecule generally reduces or inhibits the function of the target ps20 polynucleotide. Reduction in expression of a ps20 polypeptide may be achieved by the administration of antisense nucleic acids or peptide nucleic acids comprising sequences complementary to those of the mRNA that encodes the ps20 polypeptide. [See the following for reviews of antisense technology and its applications: (Phillips, M. I. (ed.) Antisense Technology, Methods Enzymol., 313 and 314: 2000, and references mentioned therein; and Crooke, S. “Antisense Drug Technology: Principles, Strategies, and Applications” (1.sup.st Edition) Marcel Dekker; and references cited therein.

In aspects of the invention, a modulator of a ps20 polynucleotide is an interfering RNA (siRNA). RNA interference (RNAi) is a mechanism of post-transcriptional gene silencing modulated by double-stranded RNA (dsRNA), which is distinct from antisense and ribozyme-based approaches (see Jain, Pharmacogenomics 5: 239-42, 2004 for a review of RNAi and siRNA). RNA interference is useful in a method for treating a viral disease in a mammal by administering to the mammal a ps20 polynucleotide (e.g., dsRNA) that hybridizes under stringent conditions to a ps20 polypeptide, and attenuates expression of the ps20 polynucleotide. RNAi is mediated by short interfering RNAs (siRNA), which generally comprises a double-stranded region approximately 19 nucleotides in length with 1-2 nucleotide 3′ overhangs on each strand, resulting in a total length of between approximately 21 and 23 nucleotides. dsRNA longer than about 30 nucleotides typically induces nonspecific mRNA degradation in mammalian cells. The presence of siRNA in mammalian cells results in sequence-specific gene silencing.

siRNAs may downregulate gene expression by transferring the siRNA into mammalian cells by methods such as transfection, electroporation, or microinjection, or when expressed in cells via any of a variety of plasmid-based approaches. [See the following for reviews of RNA interference using siRNA: Tuschl, Nat. Biotechnol. 20: 446-448, 2002; See also Yu, J., et al., Proc. Natl. Acad. Sci., 99: 6047-6052, 2002; Sui, et al., Proc. Natl. Acad. Sci USA. 99: 5515-5520, 2002; Paddison, et al., Genes and Dev. 16: 948-958, 2002; Brummelkamp, et al., Science 296: 550-553, 2002; Miyagashi, et al., Nat. Biotech. 20: 497-500, 2002; Paul, et al., Nat. Biotech. 20: 505-508, 2002]. A siRNA can comprise two individual nucleic acid strands or a single strand with a self-complementary region capable of forming a hairpin (stem-loop) structure. Variations in structure, length, number of mismatches, size of loop, identity of nucleotides in overhangs, etc., can be introduced into a siRNA to trigger effective siRNA gene silencing. In aspects of the invention, the siRNA targets exons rather than introns. In other aspects of the invention, a siRNA may comprise sequences complementary to regions within the 3′ portion of the target transcript.

siRNAs employed in the present invention include RNA strands containing two complementary elements that hybridize to one another to form a stem, a loop, and optionally an overhang, preferably a 3′ overhang. In aspects of the invention, the stem is approximately 19 base pairs long, the loop is about 1-20, more preferably about 4-10, and most preferably about 6-8 nt long and/or the overhang is about 1-20, and more preferably are enzymatic RNA molecules that catalyze the specific cleavage of RNA. In certain aspects, the stem is at least 19 nucleotides in length and can be up to approximately 29 nucleotides in length. In particular aspects of the invention, the loops comprise 4 nucleotides or greater which are less likely to be subject to steric constraints than are shorter loops. An overhang can include a 5′ phosphate and a 3′ hydroxyl and may optionally comprise a plurality of U residues, for example about 1 and 5 U residues. Classical siRNAs trigger degradation of mRNAs to which they are targeted to thereby reduce the rate of protein synthesis. In addition to classical siRNAs, certain siRNAs bind to the 3′ UTR of a template transcript and can inhibit expression of a protein encoded by the template transcript by reducing translation of the transcript rather than decreasing its stability. These RNAs are referred to as microRNAs (mRNAs) which can be between about 20 and 26 nucleotides in length, e.g., 22 nt in length. mRNAs may be derived from larger precursors known as small temporal RNAs (stRNAs) or mRNA precursors, which are typically approximately 70 nt long with an approximately 4-15 nt loop. (For example, see Grishok, et al., Cell 106: 23-24; 2001; Hutvagner, et al., Science 293: 834-838, 2001; Ketting, et al., Genes Dev., 15: 2654-2659, 2001). MicroRNAs have been found to block translation of target transcripts containing target sites in mammalian cells (Zeng, et al., Molecular Cell 9: 1-20, 2002).

In an embodiment, the invention provides a method of inhibiting expression of a gene encoding a ps20 polypeptide comprising the step of (i) providing a biological system in which expression of a gene encoding a ps20 polypeptide is to be inhibited; and (ii) contacting the system with a siRNA targeted to a transcript encoding ps20 polypeptide. In embodiments of the invention, the biological system comprises a cell (e.g., CD4 T cells), and the contacting step comprises expressing the siRNA in the cell. In other embodiments, the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

A variety of RNA molecules containing duplex structures may be employed to mediate silencing of ps20 polynucleotides through various mechanisms. Any such RNA, one portion of which binds to a target transcript and reduces its expression, whether by triggering degradation, by inhibiting translation, or by other means, is considered to be an siRNA, and any structure that generates such an siRNA is useful in the practice of the present invention.

Hairpin structures that mimic siRNAs and mRNA precursors may be processed intracellularly into molecules capable of reducing or inhibiting expression of target ps20 transcripts (for example, see McManus, et al., RNA 8: 842-850, 2002). These structures which are based on classical siRNAs comprising two RNA strands forming a 19 base pair duplex structure are classified as class I or class II hairpins. Class I hairpins incorporate a loop at the 5′ or 3′ end of an antisense siRNA strand (i.e., the strand complementary to the target transcript whose inhibition is desired) but are otherwise identical to classical siRNAs. Class II hairpins resemble mRNA precursors and comprise a 19 nt duplex region and a loop at either the 3′ or 5′ end of the antisense strand of the duplex in addition to one or more nucleotide mismatches in the stem. Hairpins are processed intracellularly into small RNA duplex structures capable of mediating silencing.

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a ps20 polypeptide. Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

In aspects of the invention, a composition is provided for treating a patient suffering from, or who may be susceptible to a viral disease, comprising a therapeutically effective amount of an inhibitor of a ps20 polypeptide, or substance selected in accordance with the methods of the invention including antibodies or binding agents, and a carrier, diluent, or excipient. A composition of the invention can contain at least one inhibitor of a ps20 polypeptide, or substance identified in accordance with the methods of the invention, alone or together with other active substances. The compositions of the invention may be administered together with or prior to administration of other biological factors that have been found to affect reduce or inhibit viral diseases.

A composition of the invention contains a therapeutically effective dose of an inhibitor, for example, an amount sufficient to lower levels of ps20 polypeptide to normal levels is about 1 to 1000, 1 to 500, 1 to 250, 1 to 200, 1 to 150, 1 to 100 or 1 to 50 μg/kg/day. A method of the invention for treating and/or preventing a viral infection may involve a series of administrations of the composition. Such a series may take place over a period of 7 to about 21 days and one or more series may be administered. The composition may be administered initially at the low end of the dosage range and the dose will be increased incrementally over a preselected time course.

An inhibitor of a ps20 polypeptide, or a substance identified in accordance with a methods of the invention may be administered by gene therapy techniques using genetically modified cells or by directly introducing genes encoding the inhibitors or stimulators into cells (e.g., T cells) in vivo. Cells may be transformed or transfected with a recombinant vector (e.g. retroviral vectors, adenoviral vectors and DNA virus vectors). Genes encoding inhibitors or stimulators, or substances may be introduced into cells of a subject in vivo using physical techniques such as microinjection and electroporation or chemical methods such as coprecipitation and incorporation of DNA into liposomes. Antisense molecules may also be introduced in vivo using these conventional methods.

One or more ps20 polypeptides or polynucleotides may be targets for immunotherapy. Immunotherapeutic methods include the use of antibody therapy, in vivo vaccines, and ex vivo immunotherapy approaches. In one aspect, the invention provides one or more antibodies specific for one or more ps20 polypeptides that may be used to treat a viral disease associated with the marker. In particular, the viral disease is HIV or influenza, and one or more ps20 polypeptide antibodies may be used systemically to treat such disease.

Thus, the invention provides a method of treating a patient susceptible to, or having a viral disease that expresses one or more ps20 polypeptide comprising administering to the patient an effective amount of an antibody that binds specifically to one or more ps20 polypeptide.

The invention encompasses administration of antibodies or fragments thereof that immunospecifically bind to and antagonize ps20 polypeptides. In an embodiment, the antibody binds to a WAP domain of a ps20 polypeptide and, preferably, also antagonizes ps20 polypeptides. In other embodiments, the antibodies inhibit or reduce permissiveness of CD4 T cells or other cells or rescue or destroy CD4 T cells or other cells that are susceptible to viral infection. In another embodiment, the antibody binds to a ps20 polypeptide or domain or fragment thereof, preferably with a K_{off} of less than 3×10⁻³ to 10×10⁻³.

One or more ps20 polypeptide antibodies may also be used in a method for selectively inhibiting or killing CD4 T cells (e.g. CD45RO+/CD28+/CD57⁻ cells) or other cells secreting one or more ps20 polypeptide comprising reacting one or more ps20 polypeptide antibody immunoconjugate or immunotoxin with the cell in an amount sufficient to inhibit or kill the cell. By way of example, unconjugated antibodies to ps20 polypeptides may be introduced into a patient such that the antibodies bind to ps20 polypeptides expressed by CD4 T cells or other cells. In addition to unconjugated antibodies to ps20 polypeptides, one or more ps20 polypeptide antibodies conjugated to therapeutic agents (e.g. immunoconjugates) may also be used therapeutically to deliver the agent directly to one or more ps20 polypeptide expressing T cells and thereby destroying the cells. Examples of such agents include abrin, ricin A, Pseudomonas exotoxin, or diphtheria toxin.

In the practice of a method of the invention, ps20 polypeptide antibodies capable of inhibiting or killing CD4 T cells or other cells expressing ps20 polypeptides are administered in a therapeutically effective amount to patients with a viral disease. The invention may provide a specific and effective treatment for a viral disease. The antibody therapy methods of the invention may be combined with other therapies.

Ps20 polypeptide antibodies useful in treating a viral disease include those that are capable of initiating a potent immune response against the disease and those that are capable of direct cytotoxicity. In this regard, ps20 polypeptide antibodies may elicit cell lysis by either complement-mediated or antibody-dependent cell cytotoxicity (ADCC) mechanisms, both of which require an intact Fc portion of the immunoglobulin molecule for interaction with effector cell Fc receptor sites or complement proteins.

Ps20 polypeptide antibodies that exert a direct biological effect on cells expressing ps20 polypeptides may also be useful in the practice of the invention. Such antibodies may not require the complete immunoglobulin to exert the effect. The mechanism by which a particular antibody exerts an effect may be evaluated using any number of in vitro assays designed to determine ADCC, antibody-dependent macrophage-mediated cytotoxicity (ADMMC), complement-mediated cell lysis, and others known in the art.

The methods of the invention contemplate the administration of single ps20 polypeptide antibodies as well as combinations, or “cocktails”, of different individual antibodies such as those recognizing different epitopes of other markers. Such cocktails may have certain advantages inasmuch as they contain antibodies that bind to different epitopes of ps20 markers. Such antibodies in combination may exhibit synergistic therapeutic effects. In addition, the administration of one or more ps20 polypeptide specific antibodies may be combined with other therapeutic agents, including but not limited to antibiotics. ps20 polypeptide specific antibodies may be administered in their “naked” or unconjugated form, or may have therapeutic agents conjugated to them.

The ps20 polypeptide specific antibodies used in the present invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington: The Science and Practice of Pharmacy. (21st Edition, Popovich, N (eds), Advanced Concepts Institute, University of the Sciences in Philadelphia, Philadelphia, Pa. 2005).

One or more ps20 polypeptide specific antibody formulations may be administered via any route capable of delivering the antibodies to the disease site Routes of administration include, but are not limited to, intravenous, intraperitoneal, intramuscular, intradermal, and the like. Antibody preparations may be lyophilized and stored as a sterile powder, preferably under vacuum, and then reconstituted in bacteriostatic water containing, for example, benzyl alcohol preservative, or in sterile water prior to injection. Treatment will generally involve the repeated administration of the antibody preparation via an acceptable route of administration such as intravenous injection (IV), at an effective dose.

Dosages will depend upon various factors generally appreciated by those of skill in the art, including the type of disease and the severity, stage of the disease, the binding affinity and half life of the antibodies used, the degree of ps20 polypeptide expression in the patient, the extent of ps20 markers, the desired steady-state antibody concentration level, frequency of treatment, and the influence of any therapeutic agents used in combination with the treatment method of the invention. Daily doses may range from about 0.01 to 500 mg/kg, 0.1 to 200 mg/kg, or 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve disease inhibition or regression. Direct administration of one or more ps20 polypeptide antibodies is also possible and may have advantages in certain situations.

Patients may be evaluated for serum ps20 polypeptides and ps20 polynucleotides in order to assist in the determination of the most effective dosing regimen and related factors. Conventional assay methods may be used for quantitating circulating ps20 polypeptide levels in patients prior to treatment. Such assays may also be used for monitoring throughout therapy, and may be useful to gauge therapeutic success in combination with evaluating other parameters such as serum levels of ps20 markers.

The invention further provides vaccines formulated to contain one or more ps20 polypeptide or fragment thereof. In an embodiment, the invention provides a method of vaccinating an individual against one or more ps20 polypeptide comprising the step of inoculating the individual with the marker or fragment thereof that lacks activity, wherein the inoculation elicits an immune response in the individual thereby vaccinating the individual against the marker.

Viral gene delivery systems may be used to deliver one or more ps20 polynucleotides or ps20 polypeptides. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding one or more ps20 polypeptide or fragment thereof introduced into the patient (e.g., intramuscularly) to induce a response.

Anti-idiotypic ps20 polypeptide specific antibodies can also be used in therapy as a vaccine for inducing an immune response. The generation of anti-idiotypic antibodies is well known in the art and can readily be adapted to generate anti-idiotypic ps20 polypeptide specific antibodies that mimic an epitope on one or more ps20 polypeptides (see, for example, Wagner et al., 1997, Hybridoma 16: 33-40; Foon et al., 1995, J Clin Invest 96: 334-342). Such an antibody can be used in anti-idiotypic therapy as presently practiced with other anti-idiotypic antibodies directed against antigens associated with disease.

Genetic immunization methods may be utilized to generate prophylactic or therapeutic humoral and cellular immune responses. One or more DNA molecules encoding ps20 polypeptides, constructs comprising DNA encoding one or more ps20 markers/immunogens and appropriate regulatory sequences may be injected directly into muscle or skin of an individual, such that the cells of the muscle or skin take-up the construct and express the encoded ps20 markers/immunogens. The ps20 markers/immunogens may be expressed as cell surface proteins or be secreted. Expression of one or more ps20 markers results in the generation of prophylactic or therapeutic humoral and cellular immunity against a viral disease. Various prophylactic and therapeutic genetic immunization techniques known in the art may be used.

In another aspect, the invention provides methods for selectively inhibiting expression of ps20 polypeptide by reacting any one or a combination of the immunoconjugates of the invention with the T cells secreting ps20 polypeptides in an amount sufficient to inhibit ps20 activity.

Vectors derived from retroviruses, adenovirus, herpes or vaccinia viruses, or from various bacterial plasmids, may be used to deliver polynucleotides encoding ps20 polypeptides to a targeted site. Methods well known to those skilled in the art may be used to construct recombinant vectors that will express antisense polynucleotides for ps20 polypeptides. (See, for example, the techniques described in Sambrook et al (supra) and Ausubel et al (supra)).

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

The therapeutic activity of compositions and agents/compounds identified using a method of the invention and may be evaluated in vivo using a suitable animal model.

The invention is now described by way of numbered paragraphs
1. A method for assessing the potential efficacy of a test agent for inhibiting a viral infection in a subject, the method comprising comparing: (a) levels of one or more ps20 polypeptides or ps20 polynucleotides, in a first sample obtained from a subject and exposed to the test agent, and (b) levels of the ps20 polypeptides or ps20 polynucleotides in a second sample obtained from the subject, wherein the sample is not exposed to the test agent, wherein a significant difference in the levels of expression of the ps20 polypeptides or ps20 polynucleotides in the first sample, relative to the second sample, is an indication that the test agent is potentially efficacious for inhibiting the viral infection in the subject.
2. A method of assessing the efficacy of a therapy for inhibiting a viral infection in a subject, the method comprising comparing: (a) levels of one or more ps20 polypeptides or ps20 polynucleotides in a first sample obtained from the subject; and (b) levels of the ps20 polypeptides or ps20 polynucleotides in a second sample obtained from the subject following therapy, wherein a significant difference in the levels of expression of the ps20 polypeptides or ps20 polynucleotides in the second sample, relative to the first sample, is an indication that the therapy is efficacious for inhibiting the viral infection in the subject.
3. A method of selecting an agent for inhibiting a viral infection in a subject the method comprising (a) obtaining a sample comprising CD4 T cells expressing ps20 polypeptides or ps20 polynucleotides from the subject; (b) separately exposing aliquots of the sample in the presence of a plurality of test agents; (c) comparing levels of one or more ps20 polypeptides or ps20 polynucleotides in each of the aliquots; and (d) selecting one of the test agents which alters the levels of ps20 polypeptides or ps20 polynucleotides in the aliquot containing that test agent, relative to other test agents.
4. A method of inhibiting a viral infection in a subject, the method comprising (a) obtaining a sample comprising CD4 T cells from the subject; (b) separately maintaining aliquots of the sample in the presence of a plurality of test agents; (c) comparing levels of one or more ps20 polypeptides or ps20 polynucleotides in each of the aliquots; and (d) administering to the subject at least one of the test agents which reduces the levels of ps20 polypeptides or ps20 polynucleotides in the aliquot that test agent, relative to other test agents.
5. A method for treating or preventing a viral disease in a subject comprising administering to a subject in need thereof, an antagonist of a ps20 polynucleotide.
6. A method as described in numbered paragraph 5 wherein the antagonist is an antisense ps20 polynucleotide or an interfering RNA (siRNA) targeted to a transcript encoding a ps20 polypeptide.

Further Applications and Advantages

The invention contemplates therapeutic applications for viral diseases employing ps20 polypeptides, ps20 polynucleotides, and/or binding agents for the ps20 polypeptides.

The following non-limiting examples are illustrative of the present invention:

### EXAMPLE 1

### Preparation of Rabbit Polyclonal Antibodies

Rabbit polyclonal antibodies were generated to a mix of two peptides (1+2 below). Two rabbits were immunized: SPY201 and SPY202.

[SEQ ID NO. 4]
1. aa 51-66: EAGAPGGPRQPRADRC
[SEQ ID NO. 5]
2. C + 206-220: CKNVAEPGRGQQRHFQ
(as free acid)

### Further Preparations of Anti-ps20 Antibodies:

Monoclonal: 10 mg of purified recombinant human ps20 was used as an antigen. Hybridoma preparation and initial antibody screening was performed by Zymed (www.zymed.com). Primary bleeds were screened by ELISA with 96 well plate coated with purified ps20-V5-His protein at 0.15 μg/well with standard protocols. One clone 1G7A9H5 (IG7) gave a high reading, and is particularly useful in the invention.

Rabbit polyclonal 202-254: was generated by a standard protocol developed by Eurogentec Ltd (Belgium) using peptide immunisation. A 15-mer peptide covering amino acid 206-220 was predicted by a standard algorithm to be immunogenic and was used by Eurogentec Ltd to generate a polyclonal antibody in rabbits using their proprietary protocol that was affinity purified against the peptide.

### EXAMPLE 2

Polyclonal Antibody Binding to ps20 mRNA High G91 Jurkats Compared to ps20 mRNA Low EV Control.

One million cells of each population was fixed using the Fix & Perm Kit by ADG Ltd as per manufacturers instruction. 200,000 fixed cells were incubated with affinity purified rabbit anti-ps20 polyclonal antibody SPY 202/70253 at log antibody dilutions exactly as per manufacturers instructions. Cells were then washed and stained with 1/100 final FITC-conjugated F(ab)₂ fraction of swine anti-rabbit IgG. Stained cells were examined for FITC on a BD FACSClaibur & analyzed by CellQuest software. Data shows higher levels of binding of the antibody on a ps20 mRNA high G91 population compared to the ps20 low empty vector control.

### EXAMPLE 3

Rabbit Polyclonal Anti-ps20 Antibody Blocks HIV Infection of Cells that Express Endogenous ps20 (EV2) but has No Effect on a ps20 Negative Cell (H9).

200,000 cells were pre-incubated for 12 hours at various dilutions of rabbit anti-ps20 polyclonal Ab (rabbit 202/70253) or control rabbit IgG (not shown). HIV-1 X4 NL4-3 virus strain was then added at an MOI=0.01. Following overnight infection in a final volume of 250 uL, the cultures were maintained in a final volume of 1 ml with 50% medium replaced on days 5 and 10 post infection. The titre of the virus in the culture supernatant was determined by titration onto standard indicator cells using GFP under control of the HIV-1 promoter as a read-out. Data shows up to 5-fold suppression of HIV spread in EV2 (ps20+) but no significant effect on virus spread in the ps20 negative H9 population.

### EXAMPLE 4

Ps20 knockdown using small-interference (si)-RNA: CD4+ CCR5+ CXCR4+ adherent HeLa indicator cells that express β-galactosidase reporter gene under the control of an HIV LTR, (from Dr J-M Serrano) originally obtained from the NIH AIDS repository were seeded 6 hours prior to transfection at a density of 2×10⁵ per 24 well plate in DMEM+10% FCS+20 ug/ml Gentamycin. Parallel triplicate cultures were set up for HIV infection and for qRT-PCR. The following siRNA was purchased from Ambion: (www.ambion.com) siRNA 1 against ps20 sense 5′ GGUGACUCAAAGAAUGUGGtt 3′ [SEQ ID NO.: 6]; antisense 5′ CCACAUUCUUUGAGUCaCCtt 3′ [SEQ ID NO.: 7]; siRNA 2 against ps20 sense 5′GGCUCAGCAUCUUGAUAUUtt 3′ [SEQ ID NO.: 8], antisense 5′ AAUAUCAAGAUGCUGAGCCtt 3′ [SEQ ID NO.: 9]. A mitogen-activated protein kinase (MAPK) control siRNA (www1.qiagen.com) was used to confirm specificity of knockdown: sense 5′UGCUGACUCCAAAGCUCUGdT 3′ [SEQ ID NO.: 10], 5′CAGAGCUUUGGAGUCAGCAdT 3′ [SEQ ID NO.: 11]. 250 nM of each siRNA was diluted in a total volume of 100 ul of DMEM culture medium without serum then complexed with 10 ul of HiPerFect Transfection Reagent (Qiagen) for 15 min, added, and cultures topped up to 500 ul to give a final concentration of 50 nM each siRNA. Cells were cultured with a mix of the two ps20-specific siRNA or the MAPK-specific siRNA. 48 hours later cells were harvested by trypsinisation, washed and viable cells plated at 2×10⁴/well in a 48 well plate. 6 hours later X4 HIV-1 NL4-3 virus stock was added at various dilutions and cells cultured in a final volume of 500 ul. 36 hours later cell lysates were harvested using a Tropix Galacto-Star assay system as per manufacturers recommendation (Applied Biosystems). Cell debris was removed by centrifugation and lysate supernatants stored at −80. To assay for β-galactosidase, 15 ul of lysates were added to the Galacto-Star reporter gene assay system and the amount of chemiluminescence measured on a VICTOR™ light 1420 Luminescence Counter, with measurements taken at peak emission, which occurs 20-30 min after beginning of reaction. For qRT-PCR measurements, parallel cultures were harvested by trypsinisation, counted and processed according to standard methods.

### Results:

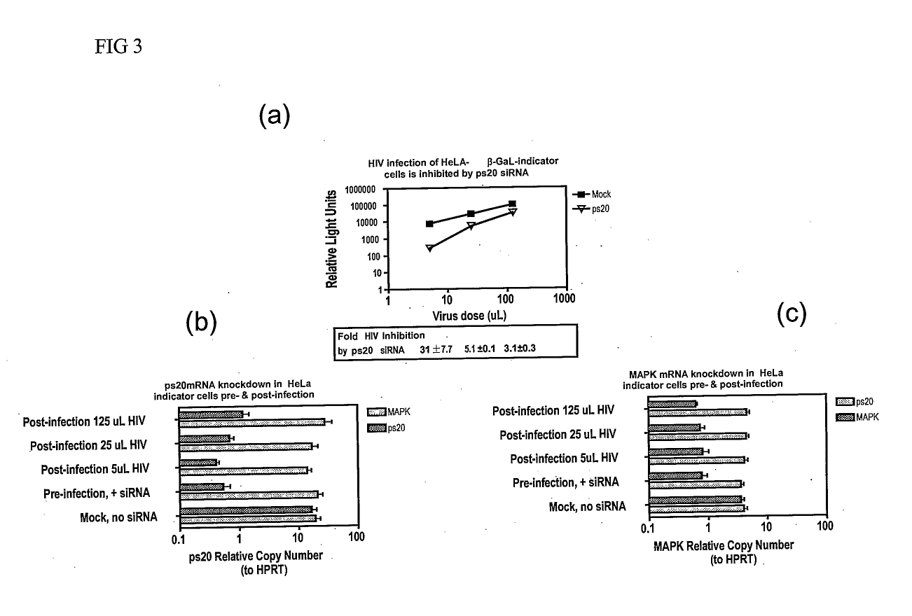

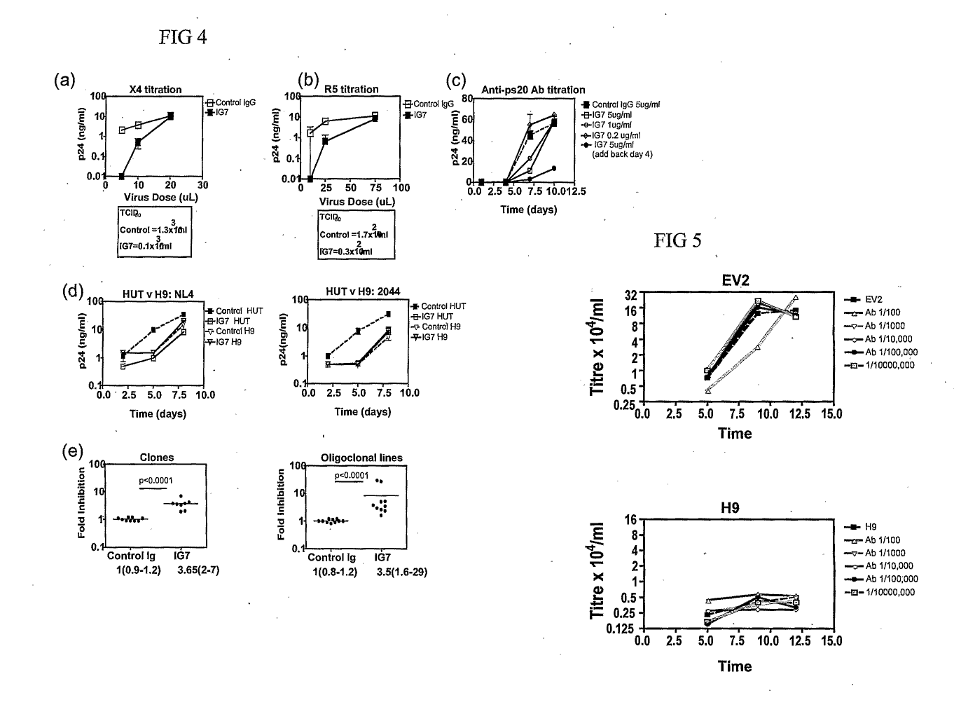

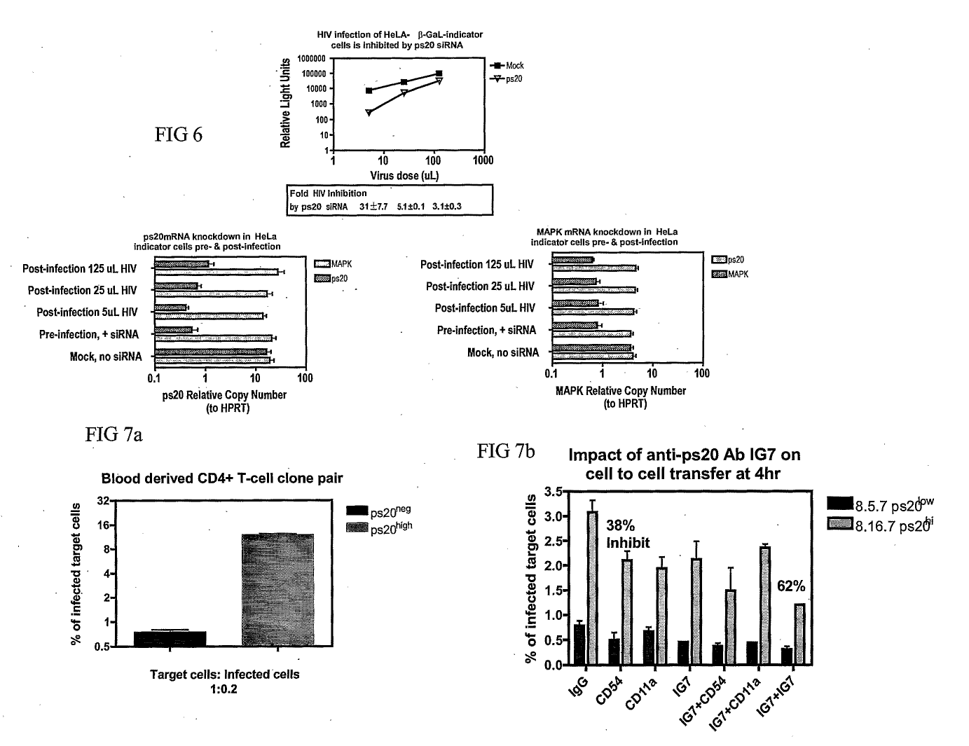

The positive acting effects of ps20 on HIV infection were illustrated employing specific siRNA in knockdown experiments using a heterologous system that is amenable to transient transfection as described below. A screen of transfectable adherent human lines identified the widely used HeLa indicator cells that express β-galactosidase reporter gene under the control of an HIV promoter to be ps20+ providing an ideal test system. Experiments were designed to correlate ps20 knockdown efficiency on HIV infection. Specificity was controlled by including another ubiquitous host gene: mitogen activated protein kinase (MAPK) mRNA. Relative to mock (transfection reagent in absence of any siRNA control) both ps20 and MAPK siRNA's were specific for their respective targets (FIG. 3b/c). ps20 Knockdown over all cultures tested ranged from 24-39-fold and for MAPF 5-7 fold with non-specific effect of each siRNA on the irrelevant target restricted to less than 1.3-fold (FIGS. 3b & 3c respectively). FIG. 3a shows a log-fold increase in infection with increasing virus input in the absence of siRNA (Mock control) and a significant and virus dose dependent inhibition of HIV infection following ps20 knockdown relative to mock. Maximum HIV inhibition of 31-fold was observed at the lowest virus dose and reduced to 3-fold inhibition at the highest virus dose despite 24-fold ps20-knockdown.

### EXAMPLE 5

### Blocking Endogenous ps20 with an Anti-ps20 Antibody Suppresses HIV Spread

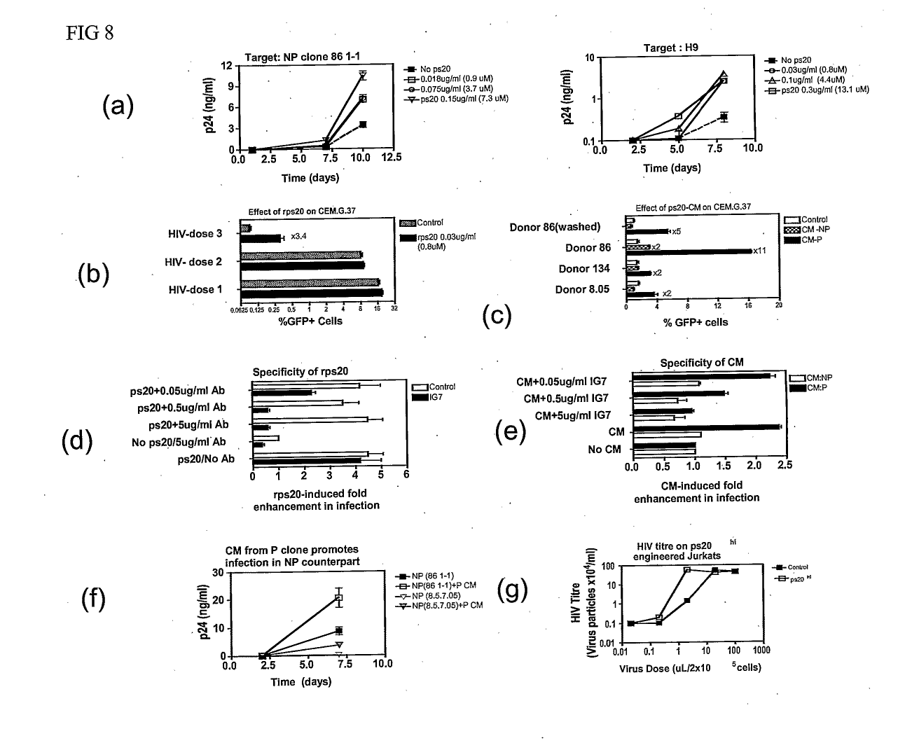

2×10⁵ ps20+ HIV permissive (P) clone 8.16.7.05 cells were pre-cultured for 18 hrs with 5 ug/ml of control mouse IgG or anti-ps20 antibody IG7, then infected with varying concentrations of X4 HIV-1 strain 2044 (FIG. 8a) or R5 HIV-1 strain YU2 (FIG. 8b) respectively in triplicate cultures. After overnight infection, cells were cultured in fresh 30 IU/ml IL2 and IG7 or control IgG in a final volume of lml. p24-CA levels were measured on day 7 post infection for 2044 and day 9 for YU2 infections and tissue culture infectious dose calculated based on the proportion of wells that were p24-CA positive for each virus dose [see 3]. Mean p24-CA levels in triplicate cultures at each virus input dose is shown. (FIG. 8c) Spreading infection in 8.16.7.05 conducted in presence of varying anti-ps20 antibody dose. p24-CA levels in triplicate cultures shown. (d) Spreading infection in H9 or HUT 78 cells conducted as in (a/b) with X4 strains (4 ng p24-CA/million cells, 2044 or NL4-3). Mean p24-CA levels in triplicate cultures shown. (e) 2×10⁵ cells (permissive clones or expanded oligoclonal CD4 lines) were pre-cultured for 18 hours with 5 ug/ml of control mouse IgG or anti-ps20 antibody IG7 or cultured in the absence of these IgG's, then infected with 2044 (2 ng p24-CA stock/million cells) for a further 18 hours. Cells were then cultured in 30 IU/ml IL2 for a further 7 days when p24-CA levels were measured. Fold inhibition in the presence of each IgG was calculated relative to p24-CA level in the absence of mouse IgG. Duplicate to triplicate measurements of two permissive clones and duplicate measurements of primary CD4 from 5 donors is shown. Group differences were determined by non-parametric Mann-Whitney test.

### EXAMPLE 6

### Anti-ps20 Rabbit Polyclonal Antibody Suppresses HIV Spread in CD4+ T Cell Cultures

200,000 cells were pre-incubated for 12 hours at various dilutions of rabbit anti-ps20 polyclonal Ab (rabbit 202/70253) or control rabbit IgG (not shown). HIV-1 X4 NL4-3 virus strain was then added at an MOI=0.01. Following overnight infection in a final volume of 250 uL, the cultures were maintained in a final volume of 1 ml with 50% medium replaced on days 5 and 10 post infection. The titre of the virus in the culture supernatant was determined by titration onto standard indicator cells using GFP under control of the HIV-1 promoter as a read-out. Data shows up to 5-fold suppression of HIV spread in EV2 (ps20+) but no significant effect on virus spread in the ps20 negative H9 population.

### EXAMPLE 7

### siRNA-Mediated Knockdown of Endogenous ps20 Suppresses HIV Spread

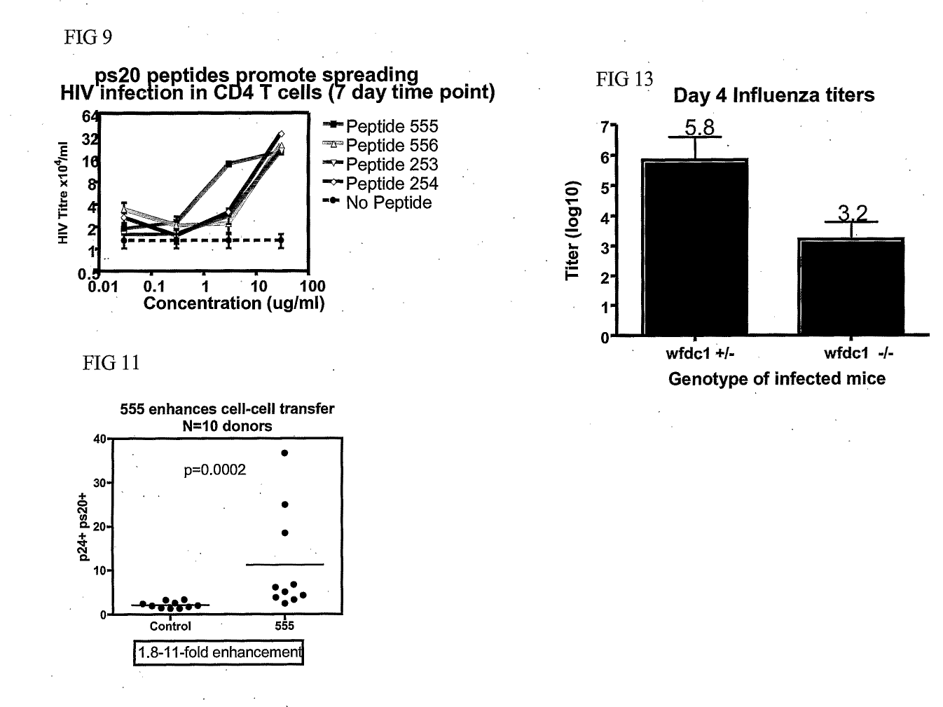

(a)2×10⁵ HeLa indicator cells were exposed to transfection reagent in absence of siRNA (mock) or 50 nM siRNA specific for ps20 or MAPK. 48 hours later, adherent cells were harvested by trypsinisation, washed and viable cells reseeded at a density of 2×10⁴ cells per well and left to adhere for 6 hours before addition of virus (5 ul, 25 ul, 125 ul). 36 hours later productive HIV infection was determined in cell lysates using b-galactosidase levels measured as relative light units (RLU) (minus background RLU by uninfected cells) in a luminometer. (b)/(c) Parallel cultures as above were set-up and samples processed for ps20 mRNA or MAPK mRNA by qRT-PCR. Non-specific effect of MAPK siRNA on ps20-knockdown is shown in FIG. 9b and vice versa of ps20 siRNA on MAPK in FIG. 9c. Error bars represent mean of three replicates.

### EXAMPLE 8

### ps20 is Important for T-T Cell-Cell Transfer of HIV

FIG. 7(a) Endogenous ps20 Promotes T-T Cell-Cell Transfer of HIV in Primary CD4 T Lymphocytes

Ps20hi (8.16.7) and ps20low (8.5.7) CD4 T cell clones were used to assess the level of cell to cell virus transfer in blood derived CD4+ T-cell clones. Jurkat CD4 T-cell cells were first infected with primary 2044 virus until the total population was 47% infected as indicated by intracellular immunofluorescence staining for HIV-1 Gag p24 antigen. These infected Jurkat cells are considered the effector population in the cell-cell transfer assay. Target cells: clone 8.5.7(ps20low) and clone 8.16.7 (ps20Hi) cells were prepared by first staining with the DDAO SE vital dye enabling these target cells to be tracked in cell mixtures. The dye positive effector cells were each then co-cultured with HIV-infected target cells at a ratio of 1:0.2 respectively. HIV transfer from the effector to target clones was measured 24 hours after the co-culture by staining for intracellular HIV Gag p24 antigen and enumerating the frequency of Gag p24 positive cells within the target dye positive population using two-colour immunofluorescence. Data shows the mean frequency of ps20hi and ps20low cells infected with HIV in three biological repeat experiments. Data shows HIV transfer to the ps20hi clone to be significantly. higher than to the ps20low clone.

FIG. 7(b) Anti-ps20 Antibody Inhibits Cell-Cell Transfer of HIV

DDAO SE vital dye labelled effectors: Ps20hi (8.16.7) and ps20low (8.5.7) CD4 T cell clones were each co-cultured with HIV-infected Jurkat target cells at a ratio of 1:0.2 effector:target cells respectively. The following conditions were examined. Anti-CD54 or antiCD11a or anti-ps20 antibody IG7 or control IgG each at 5 ug/ml final concentration was added during the co-culture and their effect assessed on virus transfer (labelled IgG/CD54/CD11a/IG7 respectively). In addition, each of the effectors was pre-cultured for 3 days with 5 ug/ml of anti-ps20 antibody and then co-cultured with targets in the presence of anti-CD54/CD11a or more IG7 (labelled CD54+IG7/CD11a+IG7 and IG7+IG7 respectively). HIV transfer from the effector to target clones was measured 24 hours after the co-culture by staining for intracellular HIV Gag p24 antigen and enumerating the frequency of Gag p24 positive cells within the target dye positive population using two-colour immunofluorescence. Data shows the mean frequency of ps20hi and ps20low cells infected with HIV in three biological repeat experiments. Preculturing the cells with anti-ps20 Ab and then maintaining it's presence during co-culture showed the highest level of inhibition of virus transfer from effector to target cell. This inhibitory effect was only observed on the ps20+ clone 8.16.7 but not on the ps20− clone 8.5.7. thereby showing specificity of effect.

### EXAMPLE 9

### Exogenous Addition of ps20 or Stable Endogenous ps20 Expression by Retroviral Transduction Promotes HIV Infection

FIG. 8(a) 2×10⁵ target cells (clone 86 1-1 or H9) were pre-cultured for 18 hours in the presence or absence (control) of recombinant (r)ps20 before infection with 2044 (1 ng p24-CA virus/per million 86 1-1 & 0.3 ng/million 1-19 cells). Mean p24 levels over time shown. FIG. 8(b) 2×10⁵ CEM.G37 indicator cells were pre-cultured for 18 hours in the presence or absence of rps20, then infected with NL4-3 (Dose 1=3 ng p24-CA stock/million cells: Dose 2=1 ng; Dose 3=0.2 ng) followed by culture for 4 days in final 1 ml volume. Mean % GFP+ cells is shown. (c) 2×10⁵ CEM.G37 cells were pre-cultured for 18 hours in the presence or absence (control) of 10% crude condition medium from NP or P clones from three donors (donor 8, donor 134 and donor 86). Cells were infected with NL4-3 (0.4 ng p24-CA stock/million cells) and maintained in the same concentration of CM. Donor 86(washed) represents cells cultured with CM prior infection, then washed, infected and maintained in the absence of CM. Mean % GFP+ cells in triplicate cultures 4 days post infection is shown. (d) 2×10⁵ NP clone 86 1-1 was pre-cultured with 1 uM rps20 alone or in presence of IG7 Ab/control IgG at varying concentrations. 18 hours later cells were infected with 2044 (1 ng p24-CA virus/per million cells) and cultures maintained in 30 IU/ml IL2 for a further 7 days. Mean fold enhancement in p24-CA levels in triplicate cultures were calculated relative to control cultures infected and maintained in the absence of any treatment. ps20/No Ab positive control set up in two sets of triplicates represented by empty and filled bars. (e) 2×10⁵ CEM.G.37 cells were pre-cultured with the most potent P CM: 2% CM from P clone 86 1-3 or counterpart NP CM in presence/absence of IG7 Ab at varying concentrations.18 hours later cells were infected with NL4-3 (0.4 ng p24-CA stock/million cells) and cultures maintained for 4 days. Mean fold enhancement in % GFP in triplicate cultures was calculated relative to control parallel cultures infected and maintained in the absence of CM plus control mouse IgG to match highest IG7 concentration tested of 5 ug/ml. (f) 2×10⁵ NP clones 86 1-1 and 8.5.7.05 were cultured for 18hours with 10% CM from P counterpart clones, then infected with 2044 (2 ng p24-CA/million cells). Cultures were maintained for 7 days. Mean p24-CA level in triplicate cultures is shown. (g 2×10⁵ Jurkat cells transduced with empty vector (EV) or ps20 (G91) were infected with varying dilutions of NL4-3 for 2 hours, washed then maintained for 7 days. HIV titre of culture supernatant was assessed on CEM G37 indicator cells using GFP expression as an indication of productive infection.

### EXAMPLE 10

### Peptide that Mimics the HIV Enhancing Effect of Recombinant Ps20 are Identified Highlighting Functional Regions of the Protein

The following peptides encoding the ps20 amino acid sequence were screened for their ability to mimic the HIV enhancing effects of ps20. A standard algorithm was used to identify two highly immunogenic epitopes. Peptides covering these epitopes are identified as 253 (amino acid {aa} 51-65 of the ps20 sequence) and peptide 254 (aa 206-220 of the ps20 sequence). In addition, two peptides that were found to bind strongly to the anti-ps20 monoclonal antibody were tested and these were identified as peptide 555 (aa 21-35) and peptide 556 (aa 91-105).

HIV infection assay: The CD4 T cell HIV-1 indicator cell line that expresses green fluorescent protein (GFP) under the control of the HIV-1 following productive HIV infection was seeded at 20,000 cells per well. Cells were cultured with varying doses of peptide between 0.2-20 ug/ml in a final volume of 200 uL for 16 hours prior to addition of varying concentrations of X4 HIV-1 NL4-3 strain. Cultures were maintained for 3 days and the percentage of GFP+ cells enumerated by standard flow cytometry analysis. Each point represents the mean GFP+cells in triplicate wells.

Peptide dose clearly influenced HIV infection. All four peptides enhanced HIV infection at 30 ug/ml. However, only the 555 peptide enhanced infection at the lower concentration of 3 ug/ml. Control culture cultures without the peptide is shown as a dotted line.

### EXAMPLE 11

### 555 ps20 Peptide Potently Mimics the HIV Enhancing Effect of Recombinant Ps20

The specificity of the ps20 peptides to enhance HIV infection was examined by comparing the effect of the peptides with peptides derived from proteins of of unrelated biology to HIV. In particular, we were keen to test the specificity of the 555 peptide and therefore the peptide dose range chosen for these studies from 20 ug/ml to 0.02 ug/ml. Controls included peptides covering the amino acid sequence of complement (identified as C34d); peptide covering the amino acid sequence of Plasmodium fakipurum and a Hirudin derived peptide HLL V. Other controls included a scrambled version of the 555 peptide and a truncated version of the 555 peptide.

HIV infection assay: The CD4 T cell HIV-1 indicator cell line that expresses green fluorescent protein (GFP) under the control of the HIV-1 following productive HIV infection was seeded at 20,000 cells per well. Cells were cultured with varying doses of peptide between 0.2-20 ug/ml in a final volume of 200 uL for 16 hours prior to addition of varying concentrations of X4 HIV-1 NL4-3 strain. Cultures were maintained for 3 days and the percentage of GFP+ cells enumerated by standard flow cytometry analysis. Each point represents the mean GFP+ cells in triplicate wells.

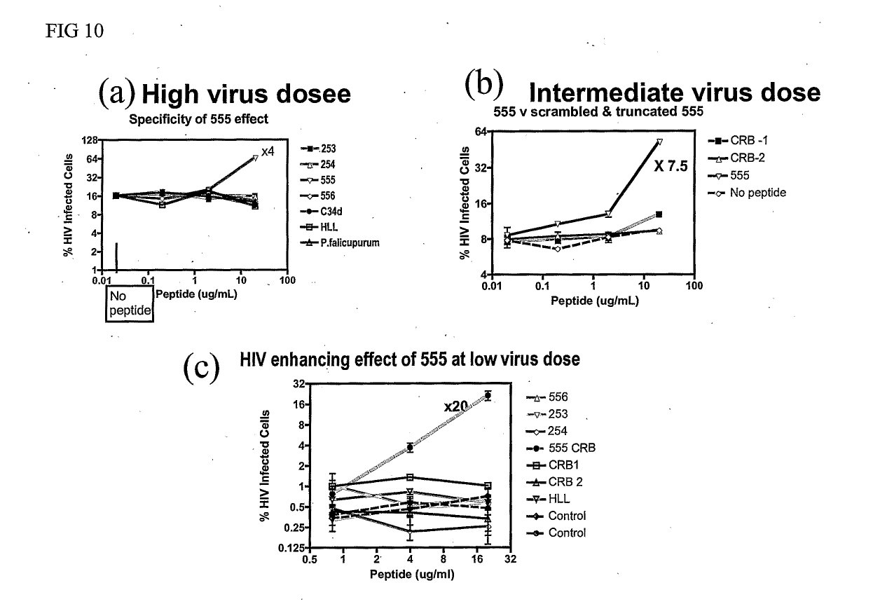

Both peptide dose and virus dose influenced HIV infection. Maximum effect was observed at 20 ug/ml peptide. In addition, the HIV enhancing effect of the peptide was higher at the lowest virus challenge dose FIG. 10(c). None of the peptides, other that 555, were effective within the dose range of 20 ug/ml to 0.02 ug/ml tested.

FIG. 10(a)HIV infection at high virus dose where the frequency of GFP+ cells in cultures without the peptide was 16%. FIG. 10(b) HIV infection at intermediate virus dose where the frequency of GFP+ cells in cultures without the peptide was 8% (0.02 ug/ml represents the no peptide control). FIG. 10(c) HIV infection at low virus dose where the frequency of GFP+ cells in cultures without the peptide labelled as “Control” is shown by dotted line was 0.5%.

### EXAMPLE 12

### 555 ps20 Peptide Enhances Cell-Cell HIV Transfer in Primary CD4 T Cells

DDAO SE vital, dye labelled CD4 T cells from ten different donors were each co-cultured with HIV-infected Jurkat target cells at a ratio of 1:1 effector:target cells respectively in the presence or absence of the 555 ps20 peptide at a final concentration of 20 ug/ml. HIV transfer from the effector to target cells was measured 24 hours after the co-culture by staining for intracellular HIV Gag p24 antigen and enumerating the frequency of Gag p24 positive cells within the target dye positive population using two-colour immunofluorescence. Data shows the frequency of target cells infected with HIV. Group comparison was using Mann-Whitney t-test.

### EXAMPLE 13

### Evidence for Immunomodulatory Role of ps20

ps20 enhances HIV infection by up-regulating cell surface cell adhesion antigen CD54, which is of known importance in promoting HIV infection.

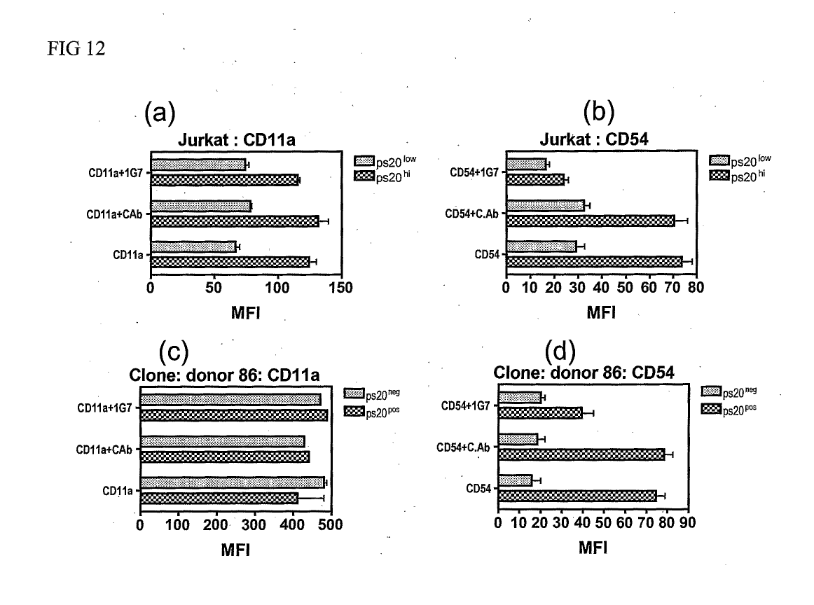

2×10⁵ cells (ps20^{low} EV v ps20^{hi} G91 Jurkats or ps20⁺ clone 86 1-3 v ps20⁻ NP clone 86 1-1) were directly stained for CD11a (FIGS. 12a & c respectively) and CD54 (FIGS. 12b & d respectively) by standard direct immunostaining and median fluorescence intensity from replicate cultures (MFI) determined. Cells were pre-cultured with either 5 ug/ml control mouse IgG or IG7 for 4 days prior staining. Untreated controls were included as indicated.

Data shows higher level of CD54 and CD11a stain on the ps20hi v ps20low Jurkats and higher CD54 but not higher CD11a on the ps20hi v ps20low CD4 T cell clone pair. Addition of anti-ps20 antibody reduced expression of CD54 but not CD11a.

### EXAMPLE 14

### ps20 is a Broad Spectrum Antiviral Target

We have generated a ps20 knock-out mouse that displays no developmental or reproductive phenotype. Upon intranasal infection of n=10 heterozygous and n=13 homozygous knock-out mice with 10 TCID₅₀ dose of influenza A/TX strain it was observed that in the absence of ps20, influenza virus replication was 3 logs lower compared to infected heterozygous mice (P=0.0013). In addition, the influenza infected ps20 knock-out mice also displayed elevated neutrophil and macrophage recruitment relative to control infected mice.

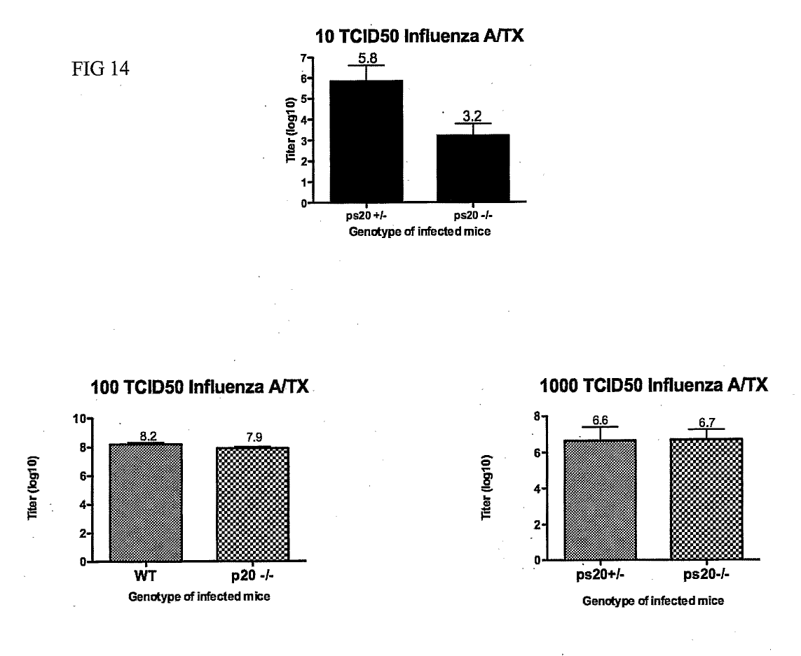

In more detail, upon infection of 10 heterozygous and 13 homozygous knock-out mice with 10TCID50 dose of influenza A/TX strain it was observed that in the absence of ps20, influenza virus replication was 3 logs lower compared to infected heterozygous mice (pvalue=0.0013). Using higher doses (1000TCID50) with 5 heterozygous and 15 homozygous knockout mice no difference in virus replication was observed (pvalue=0.97). Detailed data are shown in FIG. 14.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by. reference in their entirety to the same extent as if each individual publication, patent or patent application was specifically and individually indicated to be incorporated by reference in its entirety.

### Sequence Listing

| NM_021197 1396 bp mRNA linear | |
|---|---|
| Homo sapiens WAP four-disulfide core domain 1 (WFDC1), mRNA | |
| SEQ ID NO.: 1 | |
| 1 | agccaccatc gaggaagggg catgtgctgg acgcggacac atgatccgag ggaccctgct |
| 61 | gggtggaact aagaaagtcc agcagactgt gcatgctcct gtccccactc acaggcccac |
| 121 | gcagcgaggg gggcccctct tctgtgtgcg tctggaaggt cgctgcccag ggaggaaatg |
| 181 | cctttaaccg gcgtggggcc gggcagctgc aggaggcaga tcatccgggc tctgtgcctc |
| 241 | ttgctacttc tcctccacgc cggctctgcc aagaatatct ggaaacgggc attgcctgcg |
| 301 | aggctggccg agaaatcccg tgccgaggag gcgggcgcgc ccggcggccc ccggcagccc |
| 361 | cgagcagacc gctgcccgcc gcctccgcgg acgctgcccc ccggcgcctg ccaggccgcg |
| 421 | cgctgtcagg cggactccga gtgcccgcgg caccggcgct gctgctacaa cggatgcgcc |
| 481 | tacgcctgcc tagaagctgt gccgcccccg ccagtcttag actggctggt gcagccgaaa |
| 541 | cctcgatggc ttggtggcaa tggctggctc ctggatggcc ctgaggaggt gttacaagca |
| 601 | gaggcgtgca gcaccacgga ggatggggcc gaacccctgc tctgtccctc gggctatgag |
| 661 | tgccacatcc tgagcccagg tgacgtggcc gaaggtatcc ccaaccgtgg gcagtgcgtc |
| 721 | aagcagcgcc ggcaagcaga tgggcgaatc ctacgacaca aactttacaa agaatatcca |
| 781 | gaaggtgact caaagaatgt ggcagaacct ggaaggggac aacagaagca ctttcagtaa |
| 841 | agcaacggca agcagctagg ttgcaagaac attcctctac tttctgctaa gccttggaaa |
| 901 | cagttgggaa aagtagtttg accctcacag ttcacattca gctcagcaga gcaagacccc |
| 961 | agagatgctt agagacagga cacctggccc tcaaacccag tttggcccag cctggttggg |
| 1021 | tgactttgtg ggagccactt aacagctctg ggtccctgtt ttaccatcct gggagcaagg |
| 1081 | ccctgcagct ccacgagacc tttaccccgg gaagaagccg ccgcccatga aagcatttct |
| 1141 | gaagcccctt tctaagacaa ggctcagcat cttgatattt ttgacagatt cctcccaagt |
| 1201 | ctggctctgg gaggtatgta cccatctcaa atgttcccaa gataaattca tccttcagga |
| 1261 | aatggaaatg aacttgctta ctaatgtgtg attcctagtt gtagccaccg gatgtgctga |
| 1321 | ggcctaaatg ttagcaggtg ggaggaggcc acagaacaat aaaaacaacc aaataagaaa |
| 1381 | aaaaaaaaaa aaaaaa |
| NP_067020 220 aa linear | |
| WAP four-disulfide core domain 1 precursor [Homo sapiens]. | |
| SEQ ID NO. 2 | |
| 1 | mpltgvgpgs crrqiiralc llllllhags akniwkralp arlaeksrae eagapggprq |
| 61 | pradrcpppp rtlppgacqa arcqadsecp rhrrccyngc ayacleavpp ppvldwlvqp |
| 121 | kprwlggngw lldgpeevlq aeacsttedg aepllcpsgy echilspgdv aegipnrgqc |
| 181 | vkqrrqadgr ilrhklykey pegdsknvae pgrgqqkhfq |
| SEQ ID NO. 3 | |
| EAW95486 220 aa linear | |
| WAP four-disulfide core domain 1, isoform CRA_a [Homo sapiens] | |
| EAW95487 220 aa linear | |
| WAP four-disulfide core domain 1, isoform CRA_a [Homo sapiens] | |
| AAG16647 220 aa | |
| prostate stromal protein ps20 [Homo sapiens]. | |
| Q9HC57 220 aa linear | |
| WAP four-disulfide core domain protein 1 precursor (Prostate stromal | |
| protein ps20) (ps20 growth inhibitor). | |
| 1 | mpltgygpgs crrqiiralc llllllhags akniwkralp arlaeksrae eagapggprq |
| 61 | pradrcpppp rtlppgacqa arcqadsecp rhrrccyngc ayacleavpp ppvldwlvqp |
| 121 | kprwlggngw lldgpeevlq aeacsttedg aepllcpsgy echilspgdv aegipnrgqc |
| 181 | vkqrrqadgr ilrhklykey pegdsknvae pgrgqqrhfq |

SEQ ID NO. 4
EAGAPGGPRQPRADRC
SEQ ID NO. 5
CKNVAEPGRGQQRHFQ
SEQ ID NO. 6
5′GGUGACUCAAAGAAUGUGGtt 3′
SEQ ID NO. 7
CCACAUUCUUUGAGUCaCCtt 3′
SEQ ID NO. 8
5′GGCUCAGCAUCUUGAUAUUtt 3′
SEQ ID NO. 9
AAUAUCAAGAUGCUGAGCCtt 3′.
SEQ ID NO. 10
5′UGCUGACUCCAAAGCUCUGdT 3′
SEQ ID NO. 11
5′CAGAGCUUUGGAGUCAGCAdT

1. A method of inhibiting viral infection of a mammalian cell, said method comprising reducing or inhibiting ps20 polypeptide expressed by said cell.

2. A method according to claim 1 wherein ps20 is inhibited by contacting said cell with an antibody capable of binding to ps20 polypeptide.

3. A method according to claim 2 wherein said antibody is a ps20 neutralising antibody.

4. A method according to claim 2 wherein said antibody is a single chain antibody.

5. A composition comprising an antibody capable of binding ps20 polypeptide, siRNA targeted to a transcript encoding a ps20 polypeptide, or antisense ps20 polynucleotide wherein said composition reduces viral infection.

6. A method of treating viral infection in a subject comprising administering to said subject a composition comprising an antibody capable of binding ps20 polypeptide, siRNA targeted to a transcript encoding a ps20 polypeptide, or antisense ps20 polynucleotide in an amount effective to treat viral infection in said subject.

7. (canceled)

8. The method of claim 6, wherein said viral infection is a medicament for human immunodeficiency virus infection.

9. (canceled)

10. A method of treating or preventing viral infection in a subject, said method comprising reducing or inhibiting ps20 polypeptide in said subject.

11. A method according to claim 10 wherein said method comprises administering one or more of
(i) an antibody capable of binding to ps20 polypeptide; or
(ii) a siRNA targeted to a transcript encoding a ps20 polypeptide; or
(iii) an antisense ps20 polynucleotide
to said subject in an amount effective to reduce or inhibit ps20 polypeptide in said subject.

12. A method according to claim 10 wherein said method comprises administering anti-ps20 neutralising antibody to said subject.

13. A method according to claim 12 wherein said method comprises administering monoclonal anti-ps20 antibody IG7 to said subject.

14. A method of rendering a cell resistant to human immunodeficiency virus infection, said method comprising contacting said cell with ps20 neutralising antibody.

15. A method according to claim 10 wherein said viral infection is influenza virus or human immunodeficiency virus infection.

16. A method according to claim 15 wherein said viral infection is human immunodeficiency virus infection.

17. A method of identifying an agent for inhibiting a viral infection the method comprising
(a) providing a first and a second sample comprising CD4 T cells expressing ps20;
(b) contacting said first sample with a test agent;
(c) determining level of ps20 expression in said first and second samples; and
(d) comparing the level of ps20 expression in said first and second samples;
wherein a lower level of ps20 expression in said first sample relative to said second sample identifies said test agent as an agent for inhibiting a viral infection.

18. A method according to claim 17 further comprising the step of manufacturing a quantity of said agent so identified.

19. A method of treating a subject, said method comprising performing the method according to claim 17 wherein said first and second samples are obtained from said subject and wherein said method further comprises administering to said subject an amount of said agent so identified.

20. A method of rendering a cell permissive of viral infection, said method comprising inducing ps20 expression in said cell.

21. A method of rendering a cell permissive of viral infection, said method comprising contacting said cell with a ps20 polypeptide.

22. A method according to claim 20 or claim 21 wherein said ps20 polypeptide comprises one or more of
(i) amino acids 51-65 of the ps20 sequence;
(ii) amino acids 206-220 of the ps20 sequence;
(iii) amino acids 91-105 of the ps20 sequence; or
(iv) amino acids 21-35 of the ps20 sequence.

23. A method according to claim 22 wherein said ps20 polypeptide comprises amino acids 21-35 of the ps20 sequence.

24. A method of inducing expression of LFA-1 in a cell, said method comprising inducing ps20 expression in said cell.

25. A method of inducing expression of CD54 in a cell, said method comprising inducing ps20 expression in said cell.

26. A method according to claim 21 wherein said ps20 polypeptide comprises one or more of
(i) amino acids 51-65 of the ps20 sequence;
(ii) amino acids 206-220 of the ps20 sequence;
(iii) amino acids 91-105 of the ps20 sequence; or
(iv) amino acids 21-35 of the ps20 sequence.
